(19) United States
(12) Patent Application Publication
(10) Pub. No.: US 2011/0021609 A1
(43) Pub. Date: Jan. 27, 2011

(54) MicroRNA Signatures Associated with Cytogenetics and Prognosis in Acute Myeloid Leukemia (AML) and Uses Thereof

(75) Inventors: Carlo M. Croce, Columbus, OH (US); Ramiro Gazon, Columbus, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: THE OHIO STATE UNIVERSITY RESEARCH FOUNDATION, Columbus, OH (US)

(21) Appl. No.: 12/919,901

(22) PCT Filed: Feb. 27, 2009
(86) PCT No.: PCT/US2009/035482

§ 371 Date: Sep. 23, 2010

Related U.S. Application Data
(60) Provisional application No. 61/067,419, filed on Feb. 28, 2008.

(51) Int. Cl. A61K 48/00; C07H 21/02; C40B 30/04; C12Q 1/68; A61P 35/04; C12Q 1/02; C12N 5/00

(52) U.S. Cl. 514/44 A; 536/23.1; 506/9; 435/6; 435/29; 435/375

(57) ABSTRACT

Methods and compositions utilizing an miRNA signature for the diagnosis, prognosis and/or treatment of leukemia associated diseases, particularly acute myeloid leukemia, are disclosed.

### CROSS-REFERENCE TO RELATED APPLICATIONS

This application claims the benefit of U.S. Provisional Application No. 61/067,419, filed Feb. 28, 2008, the entire disclosure of which is expressly incorporated herein by reference.

### STATEMENT REGARDING FEDERALLY SPONSORED RESEARCH

This invention was made with government support under the NCI Grant Number(s) CA76259 and CA8134. The government has certain rights in this invention.

### TECHNICAL FIELD AND INDUSTRIAL APPLICABILITY OF THE INVENTION

This invention relates generally to the field of molecular biology. Certain aspects of the invention include application in diagnostics, therapeutics, and prognostics of leukemia related disorders.

### BACKGROUND OF THE INVENTION

There is no admission that the background art disclosed in this section legally constitutes prior art.

Acute myeloid leukemia (AML) is a cytogenetically and molecularly heterogeneous disorder characterized by differentiation arrest and malignant proliferation of clonal myeloid precursors.(1) Patients with intermediate- and poor-risk cytogenetics represent the majority of AML; chemotherapy-based regimens fail to cure most of these patients, and stem-cell transplantation is frequently the treatment of choice.(2,3) Because allogeneic stem-cell transplantation is not an option for many patients with high risk leukemia for a variety of reasons, there is a critical need to improve our understanding of the biology of these leukemias to develop novel therapies.

MicroRNAs (miRNAs) are noncoding RNAs of 19 to 25 nucleotides in length that regulate gene expression by inducing translational inhibition and cleavage of their target mRNAs through base pairing to partially or fully complementary sites.(4) miRNAs are involved in critical biologic processes, including development, cell differentiation, stress response, apoptosis, and proliferation.(4) Recently, miRNA expression has been linked to hematopoiesis and cancer.(5-11) In mice, the ectopic expression of miR-181 in hematopoietic progenitor cells led to proliferation in the B-cell compartment.(5) Likewise, important roles for miRNAs have been found during human granulocytic, erythrocytic, and megakaryocytic differentiation.(6-8) The first report linking miRNAs and cancer involved chronic lymphocytic leukemia (CLL).(9) A cluster of 2 miRNAs, miR-15a and miR-16-1, was found to be located in the minimal region of deletion (−30 kb) at 13q14 and to be deleted or down-regulated in approximately 60% of CLL samples.(9) Further studies confirmed the widespread involvement of miRNAs in cancer.(10,11) Little is known, however, about miRNA expression in AML.

In spite of considerable research into therapies to treat these diseases, they remain difficult to diagnose and treat effectively, and the mortality observed in patients indicates that improvements are needed in the diagnosis, treatment and prevention of the disease.

### SUMMARY OF THE INVENTION

In a first broad aspect, there is described herein a . . . .

Once claims are finalized, will insert summary of claims here . . . .

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIG. 1: miRNAs down-regulated in AML samples with respect to CD34⁺ cells and mature and hematopoietic precursors.

FIG. 1A, FIG. 1B: We selected the most differentiated miRNAs according to SAM score and fold change and measured them in a random group of 6 AML patients and 4 CD34 samples obtained from healthy donors by quantitative RT-PCR. Results are presented as fold change of the miRNA expression in AML samples with respect to the CD34⁺ expression from one healthy donor after normalization with let-7i and 2^{Δ}Ct conversion(18) (thin bars represent standard deviations). The difference in miRNA expression between the 4 CD34s and all the 6 AML patients was statistically significant by the t test: miR-106a (P=0.001), miR125a (P=0.001), miR-126 (P=0.001), miR-93(P=0.001), miR-130a (P=0.006), miR-146 (P=0.001), except for miR-135 (P=0.38).

FIG. 1C: Average miRNA expression (from 4 different healthy donors) of peripheral blood mature granulocytes and monocytes and bone marrow committed (erythrocytic and megakaryocytic) precursors and 6 AML patients compared with that of CD34⁺ cells after normalization and 2^{Δ} Ct conversion. The results are presented as fold change, with respect to the CD34⁺ cells, average miRNA expression. The down-regulation of miRNA expression in mature peripheral blood cells and committed precursors with respect to CD34 cells was statistically significant by t test (P<0.05).

FIG. 2: MiR-155 expression in AML with FLT3-ITD mutations. Average miR-155 expression in AML patients with FLT3-WT (n=12) and FLT3-ITD positive mutations (n=4) measured by quantitative RT-PCR. The miRNA expression between the different groups was compared using t test (SPSS).

FIGS. 3A-3B: miRNAs associated with overall survival in newly diagnosed patients with AML. Kaplan-Meier estimates of overall survival for 60 AML patients with high or low expression of miR-191 (FIG. 3A) and miR-199a (FIG. 3B) detected by quantitative RT-PCR. The log-rank test was used to compare differences between survival curves.

FIG. 4: Table 1—Clinical and cytogenetic characteristics of newly diagnosed AML patients. No statistically significant differences were observed between the two set of patients (microarrays vs. quantitative RT-PCR) by t test and x², except for the category of AML without maturation (x², P=0.03). All the values represent frequencies (%). * Those AML cases do not fulfill criteria for inclusion in one of the previously described subgroups. ^{†}Other cytogenetics groups not otherwise categorized in the WHO classification. A total of 116 of 122 patients from the microarray cohort and 59 of 60 patients from the quantitative RT-PCR cohort had at least 20 or more metaphases analyzed by conventional karyotype. Complex karyotype is defined as more than or equal to 3 chromosomal abnormalities. ^{‡}Not all the patients had FLT3 analyzed. The percentages shown are in relationship to the total number of patients with FLT3 mutation studies. ^{§}The median follow-up for alive patients in the 122 AML patients is 100 weeks (range, 1-586 weeks) and in the 60 AML cohorts is 124 weeks (range, 7-278 weeks).

FIG. 5: Table 2—MiRNAs down-regulated in 122 newly diagnosed AML patients with respect to CD34+ cells obtained from 10 healthy donors.

FIG. 6: Table 3—Influence of miRNAs on the clinical multivariate model for outcome prediction.

FIG. 7: Table S1. Housekeeping gene probes used in the normalization of microarray data (PDF, 15.2 KB).

FIG. 8: Table S2. MicroRNAs associated with WBC count and peripheral and bone marrow blast percentage (PDF, 27.5 KB). All miRNAs are up-regulated and have a positive correlation with WBC count and PB and BM blast percentage. These results were obtained by using quantitative SAM analysis. MiRNAs highlighted in yellow are shared in at least two signatures.

FIG. 9: Table S3. MicroRNAs differentially expressed in patients with t(11q23) compared with other AML patients with other cytogenetic abnormalities including normal karyotype (PDF, 19.3 KB). MiRNAs in red are up-regulated, in green down-regulated. The same signature was observed in an independent set of treated patients with t(11q23) (4) vs. other cytogenetic abnormalities (44), except for miR-196a, miR-372 and miR-193.

FIG. 10: Table S4—MicroRNAs differentially expressed between patients with t(6;11)n=4 Vs. t(9;11)5 (PDF, 17.3 KB).

FIG. 11: Table S5—MicroRNAs differentially expressed in patients with isolated trisomy 8 compared with other AML cytogenetics subgroups (PDF, 28.4 KB). For this analysis we included only samples with isolated trisomy 8. These samples were compared with other AML samples with known cytogenetics, excluding those samples with trisomy 8 as a secondary cytogenetics abnormality. All miRNAs are up-regulated.

FIG. 12: Table S6—MicroRNAs differentially expressed in normal karyotype AML patients compared with abnormal karyotype AML (PDF, 19.6 KB). All miRNAs, except miR-368, miR-191 and miR-192 were found also differentially expressed in treated AML patients with normal karyotype (10) compared with treated AML patients with abnormal karyotype (38). MiRNAs in red are up-regulated, in green down-regulated.

FIG. 13: Table S7—Clinical characteristics of 54 treated AML patient samples (relapsed n=34 or primary refractory n=20) (PDF, 68.9 KB). □—Those AML cases do not fulfill criteria for inclusion in one of the previously described subgroups. †—Other Cytogenetics groups not otherwise categorized in the WHO classification. 116 from 122 patients from the microarray cohort and 59 from 60 patients from the qRT-PCR cohort had at least 20 or more metaphases analyzed by conventional karyotype. ‡ Complex karyotype is defined as: •3 chromosomal abnormalities. *—Not all the patients had FLT3 analyzed. The percentages shown are in relationship to the total number of patients with FLT3 mutation studies (n=30).

FIG. 14: Table S8—MicroRNAs differentially expressed in treated patients with t(11q23) compared with other treated AML patients with other cytogenetic abnormalities including normal karyotype (PDF, 28.2 KB). Up-regulated red (Bold), down-regulated green (normal type).

FIG. 15—Table S9—MicroRNAs differentially expressed in normal karyotype treated AML patients compared with abnormal karyotype treated AML patients (PDF, 29.3 KB). * These miRNAs had a FDR>5. However they are shown here for comparison purposes with the signatures observed in untreated patients.

FIG. 16: Table S10—MicroRNAs up-regulated in treated AML patients with FLT3-ITD mutations vs. FLT3-wt (PDF, 13.7 KB).

FIG. 17: Validation of microarray data by qRT-PCR (JPG, 33.8 KB). Scatter plot showing the positive correlation between the miRNA microarrays expression values and the normalized qRT-PCR after 2^{Δ}Ct conversion for each sample. The solid pink line represents the predicted Y, while the blue dots are patient samples. The lower the qRT-PCR (^{Δ}Ct values), the lower the expression level of the miRNA.

FIGS. 18A-18B: Validation of the microarray results for selected miRNAs in patients with t(9;11) (JPG, 28.3 KB). Average miR-326 (FIG. 18A) and miR-29a, miR-29b and miR-29c (FIG. 18B) expression in newly diagnosed AML patients with t(9;11) (n=3) and non 11q23 AML (n=10) measured by qRT-PCR. The miRNA expression between the different groups was compared using t-test (SPSS).

FIGS. 19A-19B: Validation of the microarray results for selected miRNAs in patients with normal karyotype (JPG, 31.1 KB). Average miR-10a (FIG. 19A), miR-126 (FIG. 19B) and miR-30c (FIG. 19C) expression in newly diagnosed AML patients with normal karyotype (n=12) and abnormal karyotype (n=22) measured by qRT-PCR. The miRNA expression between the different groups was compared using t-test (SPSS).

### DETAILED DESCRIPTION OF THE PREFERRED EMBODIMENT(S)

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

A large set of AML patients with predominantly intermediate and poor prognosis was analyzed using miRNA microarrays to investigate whether miRNA expression is associated with clinical features, cytogenetic abnormalities, and outcome.

The present invention is further explained in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference.

### EXAMPLE I

### Patients and Cell Samples

Pretreatment bone marrow and blood samples from 182 patients with newly diagnosed

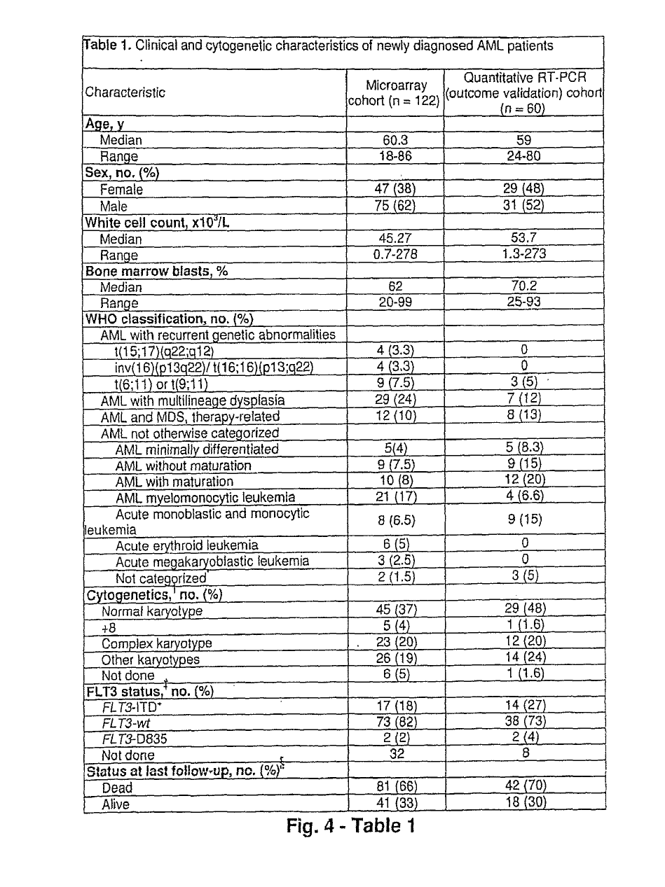

AML were obtained from the Cell and Tissue Bank at M. D. Anderson Cancer Center (MDACC; (n=172) and Thomas Jefferson University (n=10). A total of 122 AML samples were used to analyze the miRNA expression using a microarray platform, whereas 60 untreated AML samples were used to validate the outcome signatures using quantitative real-time polymerase chain reaction (RT-PCR; FIG. 4—Table 1).

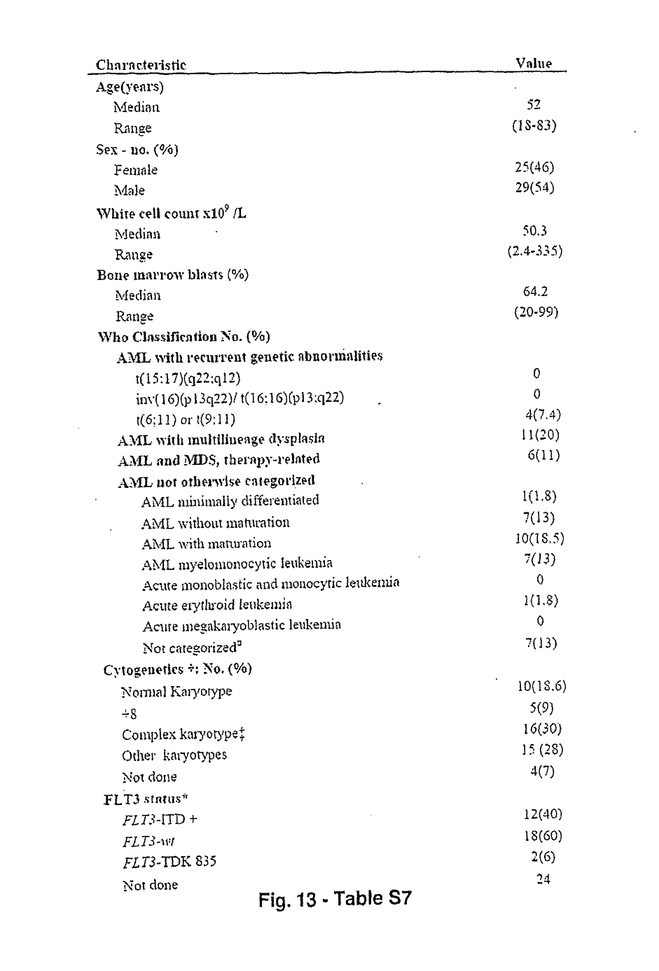

A second cohort of 54 AML patients with relapsed (n=34) or refractory (n=20) disease obtained from the MDACC was used to determine differences in the miRNA expression between newly diagnosed and relapsed/primary refractory AML patients (FIG. 13—Table S7). Informed consent was obtained from the patients in accordance with the Declaration of Helsinki to procure and bank the cells for future research according to institutional guidelines. Patient's samples were prepared by Ficoll-Hypaque (Sigma-Aldrich, St Louis, Mo.) gradient centrifugation, enriched for leukemic cells by CD3/CD19 depletion (MACS; Miltenyi Biotec, Auburn, Calif.) and cryopreserved.(12) Cytogenetic analyses of the samples were performed at diagnosis or at relapse, using unstimulated short-term (24-, 48-, and 72-hour) cultures with or without a direct method and G-banding. The criteria used to describe a cytogenetic clone and description of karyotype followed the recommendations of the International System for Human Cytogenetic Nomenclature.(13) At least 20 bone marrow metaphase cells were analyzed in patients designated as having a normal karyotype. FLT3 in tandem duplication (ITD) and activation loop D835 mutations were performed on most of the samples as previously described.(14) The first cohort of 122 AMLs was treated within a variety of institutional review board-approved protocols open at the MDACC during the collection period, including idarubicin with 2 different cytarabine combinations (n=53; protocol 91004 and 10193), high dose ARA-C (n=20) containing regimens (protocols 330139 and 202074), DCTER (n=5; protocol 202089), and investigational drugs, such as PKC 412 and interleukin-11 (n=24; protocols 201591 and 20202). All 4 patients with acute promyelocytic leukemia received regiments containing all-trans-retinoic acid. The majority of the 60 patients in the validation cohort (78%) were treated with the same idarubicin and cytarabine regimen (n=47; protocol 91003), high dose ARA-C containing regimens (n=5; protocols 330139 and 202074) and other investigational agents, such as PKC 412 and interleukin-11 (n=6; protocols 201591 and 20202). Blood mature granulocytes and monocytes and bone marrow CD71⁺ selected erythrocyte precursors from 4 healthy donors were purchased from Allcells (Emeryville, Calif.). Bone marrow CD34⁺ progenitors from 10 healthy donors were purchased from Allcells. In vitro differentiated megakaryocytes were obtained as previously described.(8)

### RNA Extraction and miRNA Microarray Experiments

RNA extraction and miRNA microchip experiments were performed as previously described.(15) The miRNA microarray is based on a one-channel system.(15) The chips contain gene-specific oligonucleotide probes, spotted by contacting technologies and covalently attached to a polymeric matrix (Example II herein ArrayExpress database at EBI for the miRNA oligonucleotide probe sequences)

### Real-Time Quantification of miRNAs

The single-tube TaqMan miRNA assays were used to detect and quantify mature miRNAs as previously described(16) using PCR 9700 Thermocycler ABI Prism 7900HT and the sequence detection system (Applied Biosystems, Foster City, Calif.). Normalization was performed with let-7-i. let-7-i was chosen because it had the lowest expression variability in the microarray patient dataset. Comparative real-time PCR was performed in triplicate, including no-template controls. Relative expression was calculated using the comparative Cₜ method.(17)

### Data Analysis

Microarray images were analyzed using GENEPIX PRO. Average values of the replicate spots of each miRNA were background subtracted; log2 transformed and normalized using a set of housekeeping genes (Table S1) and the BRB Array tools (linus.nci.nih.gov/BRB-ArrayTools.html). Absent calls were threshold to 22 (4.5 in log2 scale) before statistical analysis. This level is the average minimum intensity level detected above background in miRNA chip experiments. In 2 class comparisons (e.g., CD34 vs. AML), differentially expressed miRNAs were identified using the adjusted t test procedure within the Significance Analysis of Microarrays (SAM).(18) The SAM Excel plug-in used here calculated a score for each gene on the basis of the change in expression relative to the standard deviation of all measurements. Because this was a multiple test, permutations were performed to calculate the false discovery rate (FDR) or q value. miRNAs with FDRs lower than 5% and fold changes larger than 2 were considered for further analysis. To investigate miRNAs that correlated with quantitative variables (e.g., white cell counts), we used quantitative regression analysis within SAM. The microarray dataset was deposited at Array-Express (ebi.ac.uk/arrayexpress), array accession E-TABM-405.

Survival Analysis and Definitions

Overall survival (OS) was calculated from the time of diagnosis until the date of death (censoring for alive patients at the time of the last follow-up) and event-free survival (EFS) from the time of diagnosis until relapse or death (censoring for patients who were alive at the time of the last follow-up). In the first cohort of 122 AML patients, we used the SAM method, which involved a modified Cox proportional-hazards maximum-likelihood score, to identify a set of miRNAs whose expression significantly correlated with the duration of survival. Next we validated these miRNAs in an independent cohort of 60 newly diagnosed AML patients (FIG. 4—Table 1) using quantitative RT-PCR.

Univariate Cox proportional hazard method was used in this validation set of 60 patients to identify miRNAs associated with OS and EFS. Multivariate proportional-hazards analysis was then used to assess whether miRNAs could predict outcome independently from other factors (e.g., cytogenetics and FLT-ITD^{±}) using the R 2.4.0 software. To select best among all the multivariate models, we used the Akaike Information Criteria. Kaplan-Meier plots were used to display the association of miRNA with outcome. To generate the Kaplan-Meier plots, miRNA levels, measured by quantitative RT-PCR, were converted into discrete variables by splitting the samples into 2 classes (high and low expression, according to the median expression in the full set of samples). Survival curves were obtained for each group and compared using the log-rank test.

### Statistical Analysis

Fisher exact test, t test, and x² were used to compare baseline characteristics and average miRNA expression between groups of patients. Eleven (11) reported P values were 2-sided and obtained using the SPSS software package (SPSS 15.0 for Windows).

### RESULTS

### AML patients Reveal a Distinct Spectrum of miRNA Expression Compared with Normal CD34⁺ Progenitor Cells

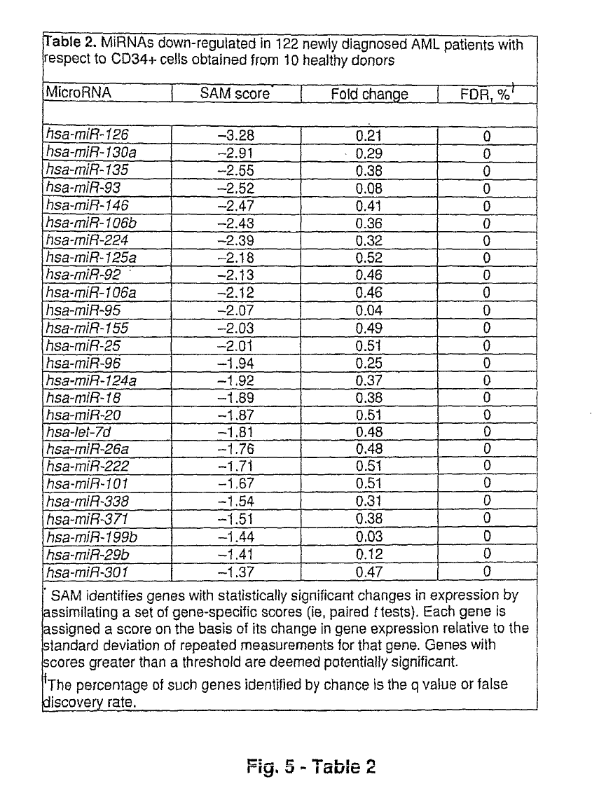

We compared 122 newly diagnosed AML samples (FIG. 4—Table 1) with CD34⁺ cells from 10 normal donors for differential miRNA expression using a previously described and validated miRNA microarray platform.(15) We identified 26 down-regulated miRNAs and none up-regulated in AML samples compared with CD34⁺ normal cells (FIG. 5—Table 2).

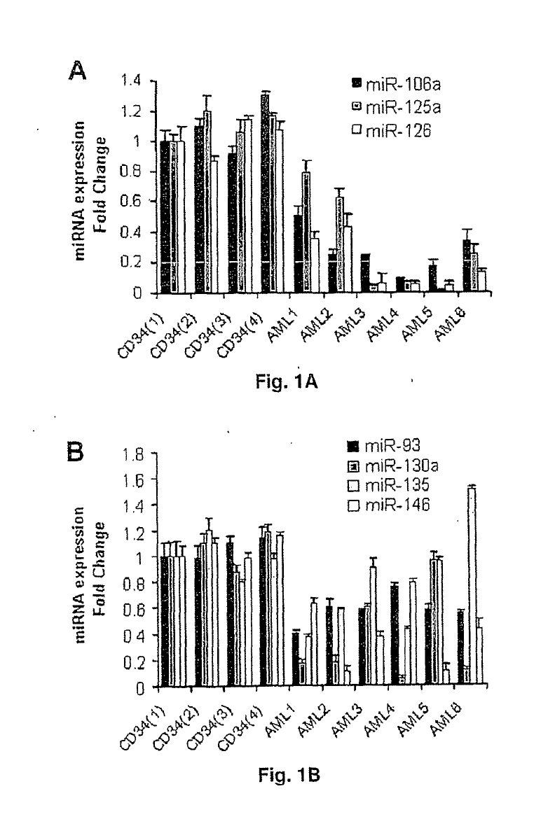

To validate these results, we performed quantitative RT-PCR for 7 of the down-regulated miRNAs (miR-126, miR-130a, miR-135, miR-93, miR-146, miR-106b, and miR-125a) using a subset of randomly chosen AML samples and 4 CD34⁺ samples obtained from different donors. As shown in FIG. 1A, FIG. 1B, we confirmed the down-regulation of the above miRNAs in AML samples with respect to the bone marrow CD34⁺ progenitors, except for miR-135.

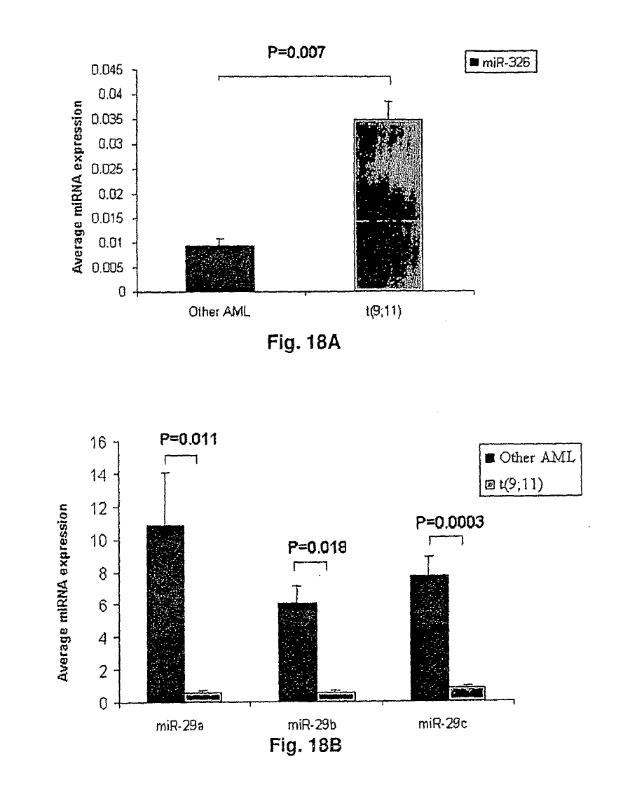

In addition, to validate the results of the microarray platform, we performed quantitative RT-PCR for 42 miRNAs whose expression was high, intermediate, and low on the chip in 12 randomly chosen AML samples. As shown in FIG. 17, the miRNA levels measured by the microarray and those measured by quantitative RT-PCR highly correlated (r=0.92, P<0.001), thereby validating the microarray platform as an analytical tool to measure miRNA expression.

### A Subset of miRNAs is Associated with Specific Hematopoietic Lineages

miRNA expression has been shown to be informative of the hematopoietic developmental lineage and differentiation stage of tumors.(11) To determine how levels of the miRNAs most differentially expressed between AML samples and CD34⁺ cells related to the different hematopoietic lineages, we assessed the expression levels of 5 of 26 miRNAs (chosen according to the SAM scores) in a panel of human hematopoietic cells, which included mature granulocytes, monocytes, and erythrocyte and megakaryocyte precursors by quantitative RT-PCR.

Among the miRNAs down-regulated in AML compared with normal CD34⁺ cells, miR-126, miR-130a, miR-93, miR-125a, and miR-146 were also significantly down-regulated in mature and precursor hematopoietic cells (FIG. 1C).

### miRNA-181a is Down-Regulated in AML with Multilineage Dysplasia

AML with multilineage dysplasia (MLD) occurs most frequently in older patients and is often associated with unfavorable cytogenetic profile and response to therapy(19) To investigate whether this group has a characteristic miRNA profile, we compared untreated AML patients with “de novo” or primary AML (n=79) with respect to AML patients with MLD (n=29) as defined by the WHO classification of AML.(19) Using SAM, we identified only the down-regulation of miR-181a in AML with MLD (FDR 0%, FC>2, SAM score of −1.68). Then, we compared untreated de novo samples (n=79) to untreated patients with therapy related AML (n=12) and identified 3 up-regulated miRNAs in therapy-related AML patients (miR-190, miR-9, and miR-188, all with FDR of 0%, FC>1.8, SAM score of >1.8). We did not detect any significant difference of miRNA expression between AML with MLD and therapy-related AML.

### miRNAs Correlate Positively to White Blood Cell and Blast Counts

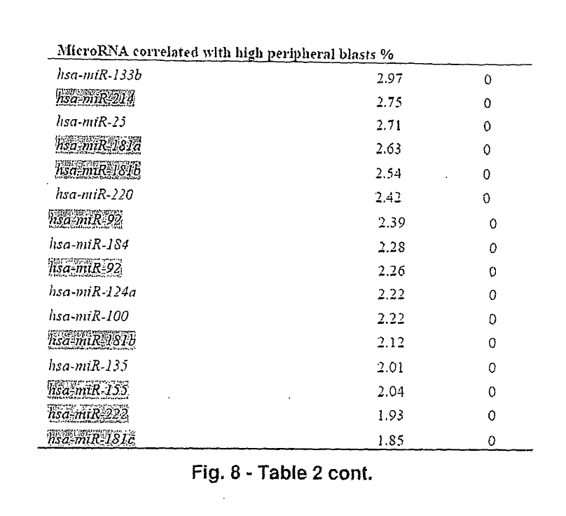

We investigated whether miRNAs are associated with pretreatment patient characteristics, such as age, sex, white blood cell (WBC) count, bone marrow, or peripheral blood blast percentage using SAM quantitative analysis as described herein. We detected a positive correlation of several miRNAs (all with FDR of 0% and high SAM quantitative scores>2), including miR-155 and miR-181b for WBC, peripheral and bone marrow blasts percentage, miR-30b and miR-30c for WBC, and bone marrow blast percentage and miR-25 for circulating blast percentages (FIG. 8—Table S2).

### miRNA Signatures Associated with Defined Cytogenetic Subgroups

To identify miRNAs associated with known cytogenetic abnormalities in AML, we studied 116 pretreatment AML samples with known karyotype. SAM was used to detect miRNAs differentially expressed between defined cytogenetic groups versus other karyotypes, including normal karyotype. Because some cytogenetics subgroups were predominantly hybridized in one batch (e.g., t(11q23) and normal karyotype), we validated the signatures using quantitative RT-PCR.

### 11q23 Balanced Translocations

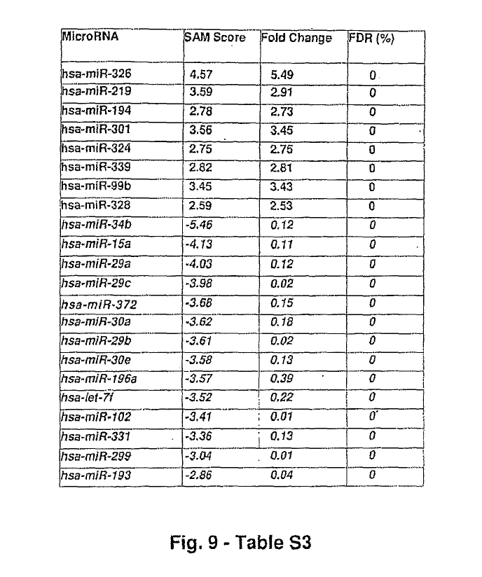

We identified 8 miRNAs up-regulated (miR-326, miR-219, miR-194, miR-301, miR-324, miR-339, miR-99b, miR-328) and 14 down-regulated (miR-34b, miR-15a, miR-29a, miR-29c, miR-372, miR-30a, miR-29b, miR-30e, miR-196a, let-7f, miR-102, miR-331, miR-299, miR-193) in patients with t(11q23) (n=9) versus all other AML patients (FIG. 9—Table S3).

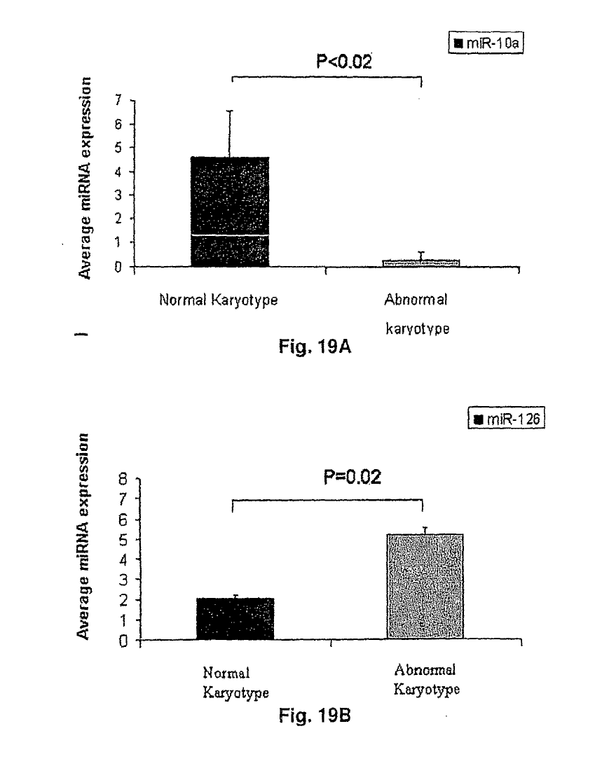

We validated the microarray results for selected miRNAs (chosen by higher SAM scores) using patient samples from the outcome validation signature cohort (non t(11q23), n=10; and t(9;11), n=3) by quantitative RT-PCR (FIGS. 18A-18B).

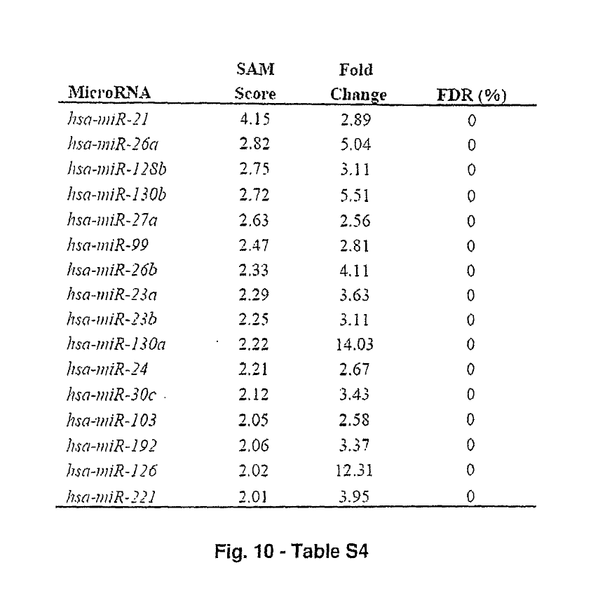

Among the miRNAs down-regulated in balanced 11q23 translocation patients, many are tumor suppressor miRNAs that target critical oncogenes, that is, miR-34b (CDK4 and CCNE2) (20), miR-15a (BCL-2) (21), the let-7 family (RAS)(22), the miR-29 family (MCL-1 and TCL-1)(23,24) miR-372 (LATS2)(25), and miR-196 (HOX-A7, HOX-A8, HOX-D8, HOX-B8)(26). Next we asked whether miRNA expression differed between patients with t(6;11) (n =4) and t(9;11) (n=5). Sixteen miRNAs were up-regulated in patients with t(6;11) (FIG. 10—Table S4), including the antiapoptotic miR-21, which targets the tumor suppressor PTEN(27) and miR-26a and b, which target the TGFb1 regulator SMAD1.(28) Down-regulation of SMAD1 has been suggested to be involved in the deregulation of TGFb1 associated with oncogenesis.(29)

### Trisomy 8

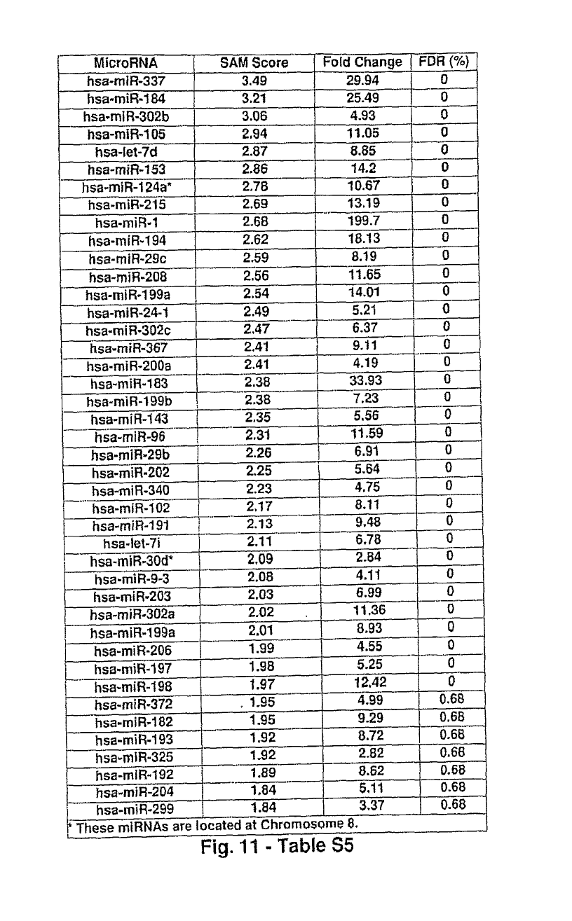

The signature obtained using SAM was comprised of 42 up-regulated and no down-regulated miRNAs in patient samples with isolated trisomy 8 (n=5) compared with all other AML patients with other karyotype after removing patients with secondary trisomy 8 (n=5; FIG. 11—Table S5).

Among the up-regulated miRNAs, miR-124a and miR-30d are located at 8p21 and 8q23, respectively, showing that a gene dosage effect may play a role in their up-regulation. Interestingly, miR-124a targets the myeloid transcription factor CEBPA.

### AML with Normal Karyotype

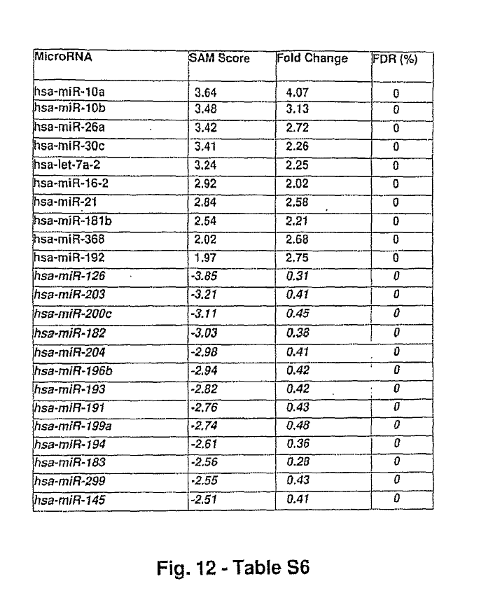

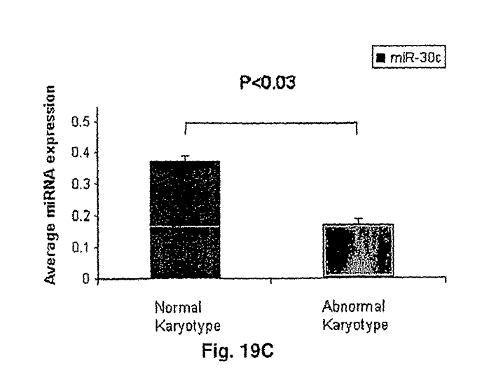

We first compared normal karyotype AML (NK-AML) patients to AML patients with abnormal karyotypes. We identified a signature in NK-AML composed of 10 up-regulated miRNAs (miR-10a, miR-10b, miR-26a, miR-30c, let-7a-2, miR-16-2, miR-21, miR-181b, miR-368, and miR-192) and 13 down-regulated miRNAs (miR-126, miR-203, miR-200c, miR-182, miR-204, miR-196b, miR-193, miR-191, miR-199a, miR-194, miR-183, miR-299, and miR-145) (FIG. 12—Table S6). This signature was not predictive of NK-AML, probably because of the molecular heterogeneity of this subgroup (data not shown). We validated the microarray results for selected miRNAs using patient samples from the outcome validation signature cohort (NK-AML, n=12; and abnormal karyotype AML, n=22 by quantitative RT-PCR; FIGS. 19A-19C).

### MiR-155 is Overexpressed in FLT3-ITD Mutations in AML Patients

To identify miRNAs associated with the presence of FLT3-ITD mutations (FLT3-ITD⁺) in AML we first compared untreated AML patients with FLT3-ITD⁺(n=17) versus FLT3-wt (n=73), excluding for FLT3-D835 mutations (n=2) using SAM. We found 3 miRNAs up-regulated in FLT3-ITD⁺, miR-155 (3.1-fold), miR-10a (2.5-fold), and miR-10b (2.27-fold), all with FDR of 0 and SAM score above 2.

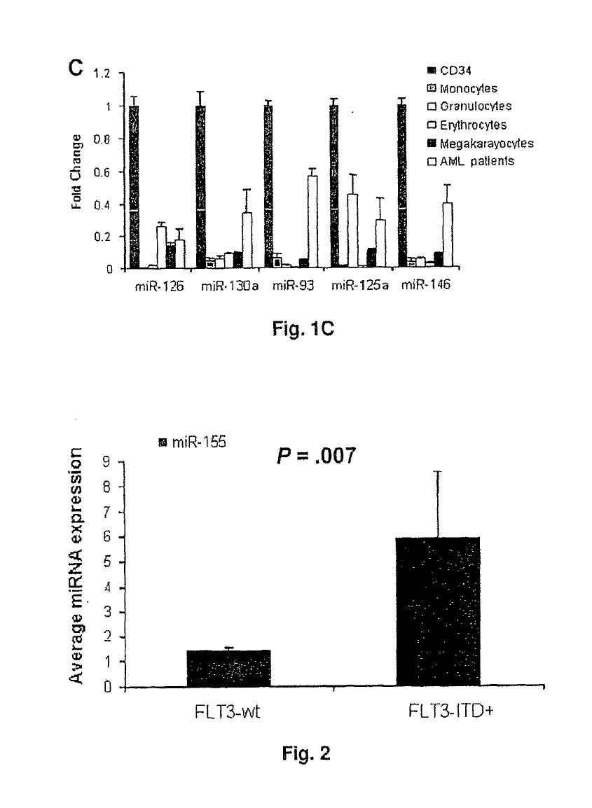

There were not enough patients with FLT3-D 835 mutations (n=2) to perform a statistical evaluation. We validated these results in an independent set of AML patients (16 patients from the outcome signature validation group) using quantitative RT-PCR. AML patients with FLT3-ITD⁺ (n=4) had again higher miR-155 expression than FLT3-wt patients (n=12, P=0.007, t test; FIG. 2).

### miRNA Expression in Relapsed and Primary Refractory AML Patients

By using our miRNA platform, we further investigated miRNA profiles of 54 patients with relapsed (n=34) or primary refractory AML (n=20; FIG. 13—Table S7).

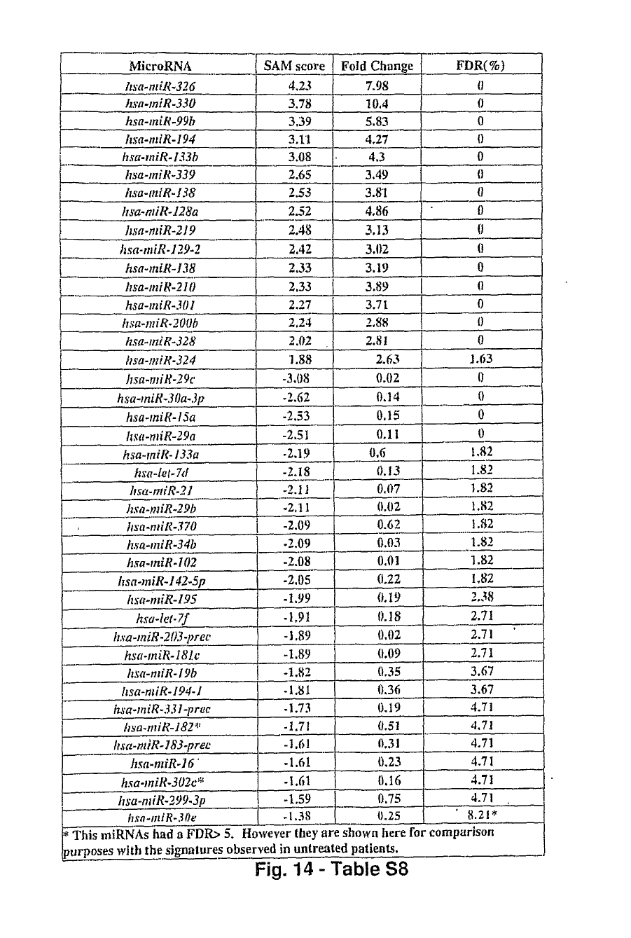

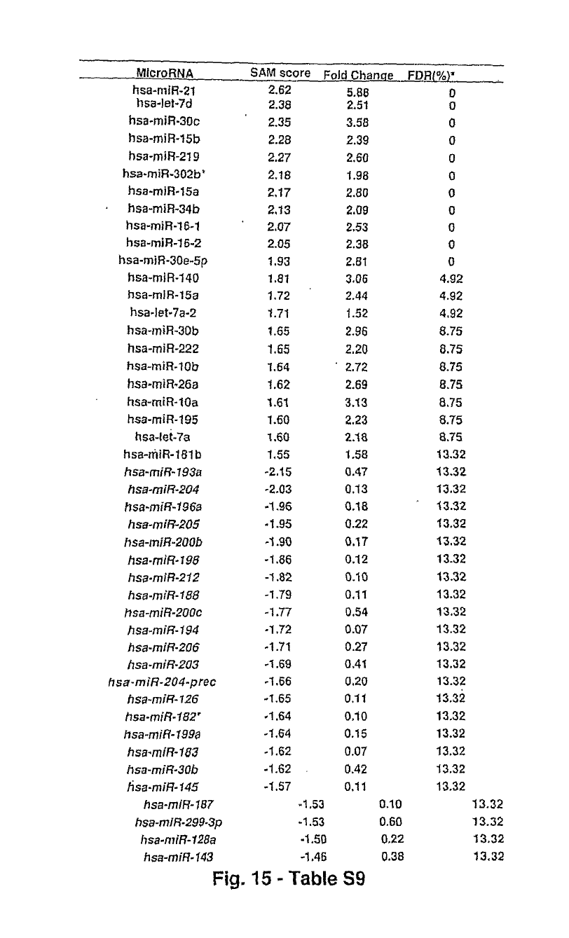

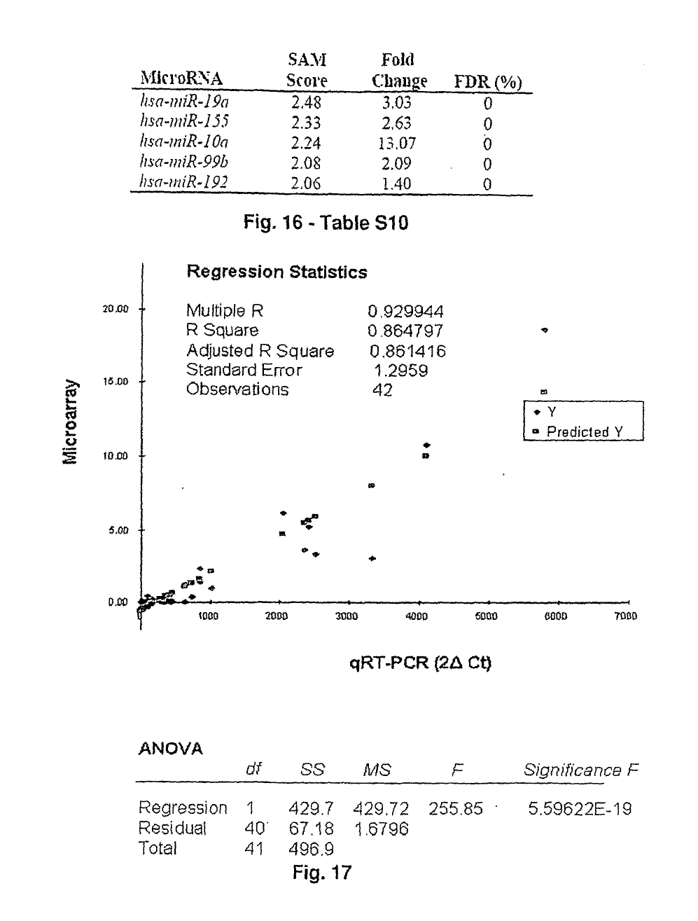

This independent cohort of treated patient samples was obtained from patients different from the initial 122 cohort. No major differences between untreated (n=122) and treated patients (n=54) were detectable (data not shown). Using this set of 54 treated patients, we analyzed miRNA expression among the different cytogenetics and molecular subgroups (e.g., AML with t(11q23) vs. other karyotypes, FLT3-ITD⁺ vs. FLT3-wt, etc) using SAM. Similar miRNAs signatures to those of the untreated patients were obtained (FIG. 14—Table S8, FIG. 15—Table 9, FIG. 16—Table S10), thereby showing that miRNA expression is largely driven by cytogenetics.

### miRNAs Associated with the Outcome

We investigated survival and miRNA expression in 122 newly diagnosed AML patients. Here, we identified a small number of miRNAs with a FDR lower than 1% and a SAM survival score (Cox regression) higher than 2. All the identified genes, miR-199a, miR-199b, miR-191, miR-25, and miR-20a, when overexpressed, adversely affected OS.

To validate this prognostic miRNA signature, we measured miR-199a, miR-191, miR-25, and miR-20a with a different technique (quantitative RT-PCR) in an independent group of 60 newly diagnosed AML patients (FIG. 4—Table 1).

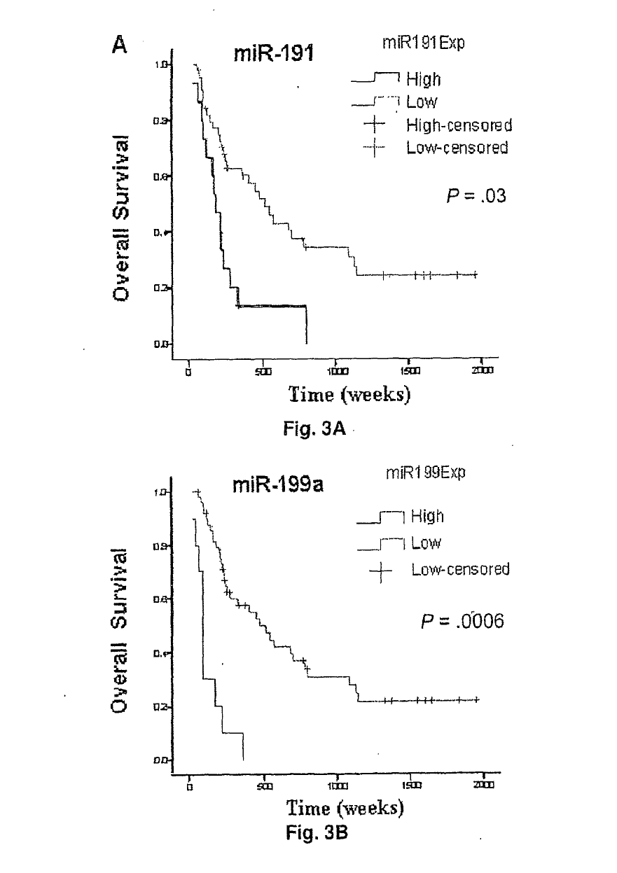

Univariate Cox proportional hazard analysis was performed to determine the association of each miRNAs to OS and EFS. We confirmed the significant associations for miR-199a and miR-191 to OS (miR-199a, P=0.001; miR-191, P=0.03) and EFS (miR-199a, P=0.002; miR-191, P=0.02). We could not validate the association of miR-20 and miR-25 to OS (miR-20 P=0.92; miR-25, P=0.07) and EFS (miR-20, P=0.8; miR-25, P=0.07). To further confirm and display graphically the association of these miRNAs with outcome, miRNA expression levels measured by quantitative RT-PCR were converted into discrete variables by splitting the samples into 2 classes (high and low expression, according to the median expression in the full set of 60 samples), and Kaplan-Meier survival plots were generated. Patients with high expression of miR-199a and miR-191 were found to have significant shorter OS (FIG. 3) and EFS (miR-199a, P=0.002; and miR-191,P=0.02, log-rank test).

Adverse cytogenetics at diagnosis, defined by the Cancer and Leukemia Group B criteria,(31) was associated with OS and EFS by univariate Cox analysis (both with P<0.001). Other characteristics, such as age (P=0.48), white blood cells (P=0.92), and FLT3-ITD⁺(P=0.2), were not significantly associated with OS nor EFS in this independent set of 60 AML patients (data not shown). The reasons behind the lack of survival association between FLT3-ITD mutations and our cohort of newly diagnosed patients may be the result of the number of patients with missing data (i.e., no FLT3 test, n=8) and the rather advanced age of the population studied (median=59 years). Contrary to young AML patients, FLT3-ITD mutations have not been found associated with poor outcome in elderly patients with AML.(32)

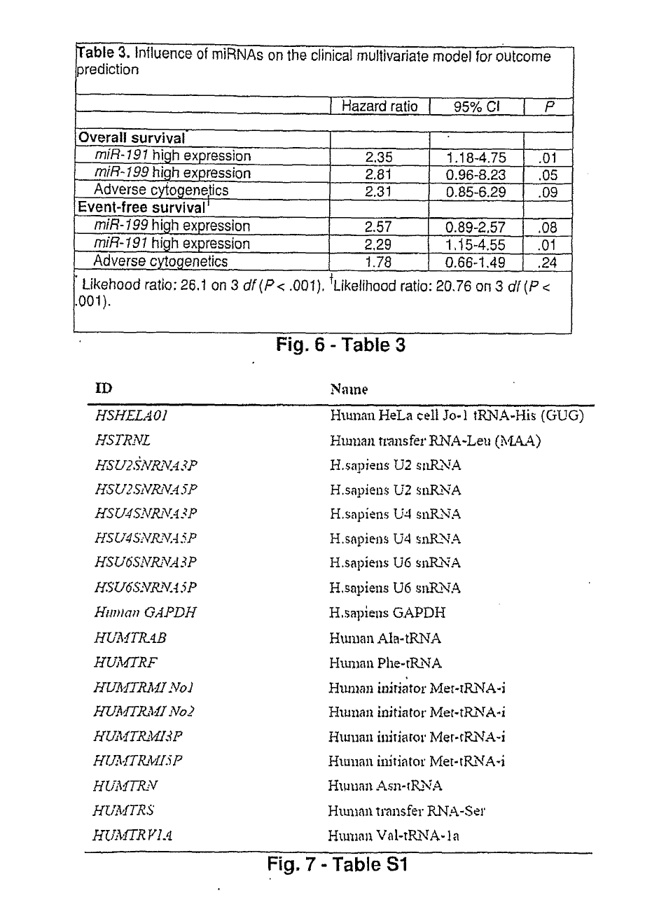

To assess whether miRNAs could predict outcome independent from other factors (e.g., cytogenetics), first we build a purely clinical model to predict OS and EFS using a Cox proportional hazard model, allowing any possible clinical covariates (WBC, FLT3-status, cytogenetics, and age). After applying the Akaike Information Criteria to eliminate redundant terms from the model, cytogenetics provided the best predictor for OS (hazard ratio=3.87; 95% confidence interval, 1.83-8.18, P<0.001) and EFS (hazard ratio=3; 95% confidence interval, 1.47-6.10, P=0.002). Then, we added the 4 miRNAs (miR-20a, miR-25, miR-191, and miR-199) as dichotomous miRNA variables (high or low miRNA expression, according to the median expression in the full set of samples) to the best clinical model. The best model keeps miR-191, miR-199, and cytogenetics for both OS and EFS (FIG. 6—Table 3).

### DISCUSSION

We used a microarray platform to perform genome wide miRNome analysis of AML samples and normal progenitor CD34⁺ cells. Most miRNAs were down-regulated in AML patients with respect to CD34⁺ cells. Two recent studies have suggested widespread miRNA down-regulation during in vitro differentiation of CD34⁺ cells to several lineages(8,33). Our data confirmed that the most down-regulated miRNAs in AML with respect to CD34⁺ cells were also down-regulated in healthy precursors and mature peripheral blood myeloid cells, showing that a subset of miRNAs in leukemia follow closely the differentiation patterns of miRNA expression in normal hematopoiesis.

Here, we identified molecular signatures associated with several cytogenetic groups. The 2 strongest signatures were those associated with balanced 11q23 translocations and isolated trisomy 8.

The down-regulation of miR-196, known to regulate HOX genes(26) in patients harboring 11q23 translocations, shows novel mechanism to explain the up-regulation of several HOX genes in these patients.

Using the microarray platform, we were also able to distinguish between t(6;11) and t(9;11). Among the up-regulated miRNAs in t(6;11), miR-21 has been found overexpressed in many solid tumors.(10) Another study indicated that miR-21 targets PTEN,(27) an important tumor suppressor, and antisense inhibition of miR-21 induces apoptosis of tumor cells in vitro and suppresses tumor growth in a xenograft mouse model.(34). Aberrant expression of oncomiRs, such as miR-21 and miR-26 in t(6;11), is now believed to explain the worse prognosis of this subgroup of patients.(31)

In contrast, miR-29 family members, down-modulated in balanced 11q23 translocations, target the oncogene TCL1(24) and MCL1(24), a critical apoptosis regulator found up-regulated in cells that are resistant to a variety of chemotherapeutic agents.(35) Moreover, other miR-29 family members are down-regulated in high risk CLL(25) and lung cancer.(37)

Interestingly, miR-155 was found to be up-regulated in AML patients with high white count and FLT3-ITD mutations. This miRNA has been recently described to block in vitro human myeloid colony formation(38), halt megakaryopoiesis(38), and induce B-cell lymphoma and leukemia in mice.(39)

There were few patients with favorable cytogenetics, such as inv(16) [4] and t(15;17) [4]. We were not able to identify any characteristic miRNA signature in these 2 groups of AML patients. The lack of correlation may be the result of heterogeneity within the groups and/or to the small sample size.

We describe a miRNA signature significantly associated with OS and EFS. Several observations strengthen our results. These subsets of miRNAs is clearly deregulated in AML and associated with cytogenetic groups and outcome.

First, we identified miRNAs associated with survival despite the overall poor prognosis and short survival of the patients studied here, where outcome differences could be difficult to demonstrate. Second, high expression of miR-199a and miR-191 was also identified in patients with isolated trisomy 8, a subgroup of AML, which is associated with poor outcome.(31) Third, the outcome signature is constituted of up-regulated miRNAs in common with the shared signatures of 6 solid tumors (e.g., miR-20, miR-25, miR-199a, and miR-191).(1)

### EXAMPLE II

### MicroRNA (miRNA) Microarrays

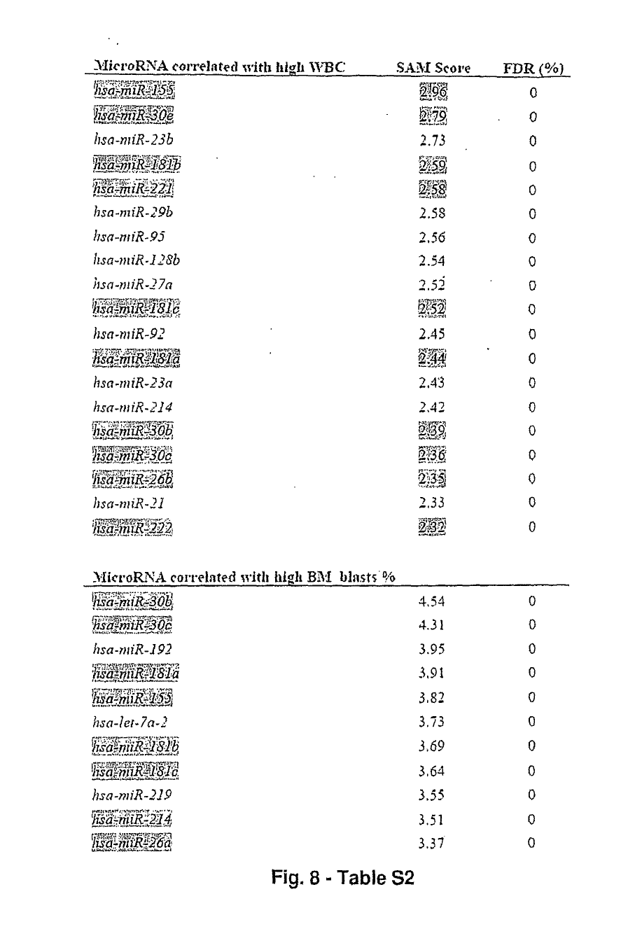

Five micrograms of total RNA was used for hybridization on the miRNA microarray chips in quadruplicate with probes corresponding to the 250 human mature and precursor miRNAs (as described in the miRBase (microrna.sanger.ac.uk) in November 2005). The total RNA was separately added to reaction mix in a final volume of 12 μl, containing 1 μg of 3′-(N)8-(A)12-biotin-(A)12-biotin-5′ random oligonucleotide primer. The mixture was incubated for 10 min at 70° C. and chilled on ice. With the mixture remaining on ice, 4 μl of 5× first-strand buffer, 2 μl of 0.1 M DTT, 1 μl of 10 mM dNTP mix, and 1 μl of SuperScript II RNaseH⁻ reverse transcriptase (200 units/μl) were added to a final volume of 20 μl, and the mixture was incubated for 90 min in a 37° C. water bath. After incubation for first-strand cDNA synthesis, 3.5 μl of 0.5 M NaOH/50 mM EDTA was added into 20 μl of first-strand reaction mix and incubated at 65° C. for 15 min to denature the RNA/DNA hybrids and degrade RNA templates. Then, 5 μl of 1 M Tris·HCl (pH 7.6, Sigma) was added to neutralize the reaction mix, and labeled targets were stored at −80° C. prior to hybridization. The microarrays were hybridized in 6×SSPE (0.9 M sodium chloride/60 mM sodium phosphate/8 mM EDTA, pH 7.4)/30% formamide at 25° C. for 18 h, washed in 0.75×TNT (Tris·HCl/sodium chloride/Tween 20) at 37° C. for 40 min, and processed by using direct detection of the biotin-containing transcripts by Streptavidin-Alexa647 conjugate. Processed slides were scanned using a GenePix Axon 4000B microarray scanner, with the laser set to 635 nm, at fixed PMT setting of 800, and a scan resolution of 10 mm. In addition to the miRNA probes, oligonucleotides for eight human TRNAs and 3 snRNAs by using similar design criteria were included. (FIG. 7—Table S1).

### Data Analysis

After obtaining the slides images using GenePix Pro, average values of the replicate spots of each miRNA were background-subtracted, normalized and subject to further analysis. Spots flagged as absent or outliers according to the GenePix Pro quality control were not included in the analysis. BRB Array Tools was used for normalization. As single-channel experiments, the arrays were normalized to a reference array, so that the difference in log-intensities between the array and reference array had median of zero over the set of housekeeping genes. The reference array was automatically chosen as the median array (the array whose median log-intensity value is the median over all median log-intensity values for the complete set of arrays). The housekeeping genes normalization was performed by computing the gene-by-gene difference between each array and the reference array, and subtracting the median difference over housekeeping genes from the log-intensities on that array. The “housekeeping” non coding genes were selected because they are non-coding as the miRNA genes (FIG. 7—Table S1).

We extended the version 1 tRNA genes to include U2, U4, U6 small non-coding RNA genes and GAPDH mRNA. U6 are extensively used in miRNA papers from different labs for normalization of Northern blots. Due to the heterogeneity of AML, the miRNAs were retained when present in at least 20% of samples. Absent calls were thresholded to 22 (4.5 in log2 scale) prior to statistical analysis. This level is the average minimum intensity level detected above background in miRNA chips experiments. MiRNA nomenclature was according to the miRNA database at Sanger Center¹. Differentially expressed miRNAs were identified by using the adjusted t test procedure within significance analysis of microarrays (SAM).² The SAM 2.0 application with a threshold difference in expression set to 2, s0 percentile set to 0.05 (default) and the number of permutations set to 100 (default). The SAM Excel plug-in used here calculates a score for each gene on the basis of the change in expression relative to the standard deviation of all measurements. Since this is a multiple test, permutations are performed to calculate the false discovery rate (FDR) or q-value. MiRNAs with FDRs less than 5% and fold changes more than 2 were considered for further analysis. The microarray dataset is deposited in Array-Express (ebi.ac.uk/arrayexpress).

### MiRNA qRT-PCR Validation

The single tube TaqMan miRNA Assays was used to detect and quantify mature miRNAs on Applied Biosystems Real-Time PCR instruments in accordance with manufacturer's instructions (Applied Biosystems, Foster City, Calif.). Normalization was performed with the invariant let-7i (Applied Biosystems). All RT reactions, including no-template controls and RT minus controls, were run in a GeneAmp PCR 9700 Thermocycler (Applied Biosystems). Gene expression levels were quantified using the ABI Prism 7900HT Sequence detection system (Applied Biosystems). Comparative real-time PCR was performed in triplicate, including no-template controls. Relative expression was calculated using the comparative Ct method.³ To validate the microarray data we used Pearson correlation and linear regression analysis (SPSS package) using 42 miRNA measurements in 12 patients. These functions examine each pair of measurements (one from the chip and the other from qRT-PCR) to determine whether the two variables tend to move together, that is whether the larger values from the chip (high expression) are associated with the higher values from the qRT-PCR (2^{Δ}Ct).

### Examples of Uses and Definitions Thereof

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

As such, the definitions herein are provided for further explanation and are not to be construed as limiting.

The articles “a” and “an” are used herein to refer to one or to more than one (i.e., to at least one) of the grammatical object of the article. By way of example, “an element” means one element or more than one element.

A “marker” and “biomarker” is a gene and/or protein and/or functional variants thereof whose altered level of expression in a tissue or cell from its expression level in normal or healthy tissue or cell is associated with a disorder and/or disease state.

The “normal” level of expression of a marker is the level of expression of the marker in cells of a human subject or patient not afflicted with a disorder and/or disease state.

An “over-expression” or “significantly higher level of expression” of a marker refers to an expression level in a test sample that is greater than the standard error of the assay employed to assess expression, and in certain embodiments, at least twice, and in other embodiments, three, four, five or ten times the expression level of the marker in a control sample (e.g., sample from a healthy subject not having the marker associated disorder and/or disease state) and in certain embodiments, the average expression level of the marker in several control samples.

A “significantly lower level of expression” of a marker refers to an expression level in a test sample that is at least twice, and in certain embodiments, three, four, five or ten times lower than the expression level of the marker in a control sample (e.g., sample from a healthy subject not having the marker associated disorder and/or disease state) and in certain embodiments, the average expression level of the marker in several control samples.

A kit is any manufacture (e.g. a package or container) comprising at least one reagent, e.g., a probe, for specifically detecting the expression of a marker. The kit may be promoted, distributed or sold as a unit for performing the methods of the present invention.

“Proteins” encompass marker proteins and their fragments; variant marker proteins and their fragments; peptides and polypeptides comprising an at least 15 amino acid segment of a marker or variant marker protein; and fusion proteins comprising a marker or variant marker protein, or an at least 15 amino acid segment of a marker or variant marker protein.

The compositions, kits and methods described herein have the following non-limiting uses, among others:
assessing whether a subject is afflicted with a disorder and/or disease state;
assessing the stage of a disorder and/or disease state in a subject;
assessing the grade of a disorder and/or disease state in a subject;
assessing the nature of a disorder and/or disease state in a subject;
assessing the potential to develop a disorder and/or disease state in a subject;
assessing the histological type of cells associated with a disorder and/or disease state in a subject;
making antibodies, antibody fragments or antibody derivatives that are useful for treating a disorder and/or disease state in a subject;
assessing the presence of a disorder and/or disease state in a subject's cells;
assessing the efficacy of one or more test compounds for inhibiting a disorder and/or disease state in a subject;
assessing the efficacy of a therapy for inhibiting a disorder and/or disease state in a subject;
monitoring the progression of a disorder and/or disease state in a subject;
selecting a composition or therapy for inhibiting a disorder and/or disease state in a subject;
treating a subject afflicted with a disorder and/or disease state;
inhibiting a disorder and/or disease state in a subject;
assessing the harmful potential of a test compound; and
preventing the onset of a disorder and/or disease state in a subject at risk therefor.

### Screening Methods

Animal models can be created to enable screening of therapeutic agents useful for treating or preventing a disorder and/or disease state in a subject. Accordingly, the methods are useful for identifying therapeutic agents for treating or preventing a disorder and/or disease state in a subject. The methods comprise administering a candidate agent to an animal model made by the methods described herein, and assessing at least one response in the animal model as compared to a control animal model to which the candidate agent has not been administered. If at least one response is reduced in symptoms or delayed in onset, the candidate agent is an agent for treating or preventing the disease.

The candidate agents may be pharmacologic agents already known in the art or may be agents previously unknown to have any pharmacological activity. The agents may be naturally arising or designed in the laboratory. They may be isolated from microorganisms, animals or plants, or may be produced recombinantly, or synthesized by any suitable chemical method. They may be small molecules, nucleic acids, proteins, peptides or peptidomimetics. In certain embodiments, candidate agents are small organic compounds having a molecular weight of more than 50 and less than about 2,500 daltons. Candidate agents comprise functional groups necessary for structural interaction with proteins. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. There are, for example, numerous means available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. In certain embodiments, the candidate agents can be obtained using any of the numerous approaches in combinatorial library methods art, including, by non-limiting example: biological libraries; spatially addressable parallel solid phase or solution phase libraries; synthetic library methods requiring deconvolution; the “one-bead one-compound” library method; and synthetic library methods using affinity chromatography selection.

In certain further embodiments, certain pharmacological agents may be subjected to directed or random chemical modifications, such as acylation, alkylation, esterification, amidification, etc. to produce structural analogs.

The same methods for identifying therapeutic agents for treating a disorder and/or disease state in a subject can also be used to validate lead compounds/agents generated from in vitro studies.

The candidate agent may be an agent that up- or down-regulates one or more of a disorder and/or disease state in a subject response pathway. In certain embodiments, the candidate agent may be an antagonist that affects such pathway.

### Methods for Treating a Disorder and/or Disease State

There is provided herein methods for treating, inhibiting, relieving or reversing a disorder and/or disease state response. In the methods described herein, an agent that interferes with a signaling cascade is administered to an individual in need thereof, such as, but not limited to, subjects in whom such complications are not yet evident and those who already have at least one such response.

In the former instance, such treatment is useful to prevent the occurrence of such response and/or reduce the extent to which they occur. In the latter instance, such treatment is useful to reduce the extent to which such response occurs, prevent their further development or reverse the response.

In certain embodiments, the agent that interferes with the response cascade may be an antibody specific for such response.

### Expression of Biomarker(s))

Expression of a marker can be inhibited in a number of ways, including, by way of a non-limiting example, an antisense oligonucleotide can be provided to the disease cells in order to inhibit transcription, translation, or both, of the marker(s). Alternately, a polynucleotide encoding an antibody, an antibody derivative, or an antibody fragment which specifically binds a marker protein, and operably linked with an appropriate promoter/regulator region, can be provided to the cell in order to generate intracellular antibodies which will inhibit the function or activity of the protein. The expression and/or function of a marker may also be inhibited by treating the disease cell with an antibody, antibody derivative or antibody fragment that specifically binds a marker protein. Using the methods described herein, a variety of molecules, particularly including molecules sufficiently small that they are able to cross the cell membrane, can be screened in order to identify molecules which inhibit expression of a marker or inhibit the function of a marker protein. The compound so identified can be provided to the subject in order to inhibit disease cells of the subject.

Any marker or combination of markers, as well as any certain markers in combination with the markers, may be used in the compositions, kits and methods described herein. In general, it is desirable to use markers for which the difference between the level of expression of the marker in disease cells and the level of expression of the same marker in normal colon system cells is as great as possible. Although this difference can be as small as the limit of detection of the method for assessing expression of the marker, it is desirable that the difference be at least greater than the standard error of the assessment method, and, in certain embodiments, a difference of at least 2-, 3-, 4-, 5-, 6-, 7-, 8-, 9-, 10-, 15-, 20-, 100-, 500-, 1000-fold or greater than the level of expression of the same marker in normal tissue.

It is recognized that certain marker proteins are secreted to the extracellular space surrounding the cells. These markers are used in certain embodiments of the compositions, kits and methods, owing to the fact that such marker proteins can be detected in a body fluid sample, which may be more easily collected from a human subject than a tissue biopsy sample. In addition, in vivo techniques for detection of a marker protein include introducing into a subject a labeled antibody directed against the protein. For example, the antibody can be labeled with a radioactive marker whose presence and location in a subject can be detected by standard imaging techniques.

In order to determine whether any particular marker protein is a secreted protein, the marker protein is expressed in, for example, a mammalian cell, such as a human cell line, extracellular fluid is collected, and the presence or absence of the protein in the extracellular fluid is assessed (e.g. using a labeled antibody which binds specifically with the protein).

It will be appreciated that subject samples containing such cells may be used in the methods described herein. In these embodiments, the level of expression of the marker can be assessed by assessing the amount (e.g., absolute amount or concentration) of the marker in a sample. The cell sample can, of course, be subjected to a variety of post-collection preparative and storage techniques (e.g., nucleic acid and/or protein extraction, fixation, storage, freezing, ultrafiltration, concentration, evaporation, centrifugation, etc.) prior to assessing the amount of the marker in the sample.

It will also be appreciated that the markers may be shed from the cells into, for example, the respiratory system, digestive system, the blood stream and/or interstitial spaces. The shed markers can be tested, for example, by examining the sputum, BAL, serum, plasma, urine, stool, etc.

The compositions, kits and methods can be used to detect expression of marker proteins having at least one portion which is displayed on the surface of cells which express it. For example, immunological methods may be used to detect such proteins on whole cells, or computer-based sequence analysis methods may be used to predict the presence of at least one extracellular domain (i.e., including both secreted proteins and proteins having at least one cell-surface domain). Expression of a marker protein having at least one portion which is displayed on the surface of a cell which expresses it may be detected without necessarily lysing the cell (e.g., using a labeled antibody which binds specifically with a cell-surface domain of the protein).

Expression of a marker may be assessed by any of a wide variety of methods for detecting expression of a transcribed nucleic acid or protein. Non-limiting examples of such methods include immunological methods for detection of secreted, cell-surface, cytoplasmic or nuclear proteins, protein purification methods, protein function or activity assays, nucleic acid hybridization methods, nucleic acid reverse transcription methods and nucleic acid amplification methods.

In a particular embodiment, expression of a marker is assessed using an antibody (e.g., a radio-labeled, chromophore-labeled, fluorophore-labeled or enzyme-labeled antibody), an antibody derivative (e.g., an antibody conjugated with a substrate or with the protein or ligand of a protein-ligand pair), or an antibody fragment (e.g., a single-chain antibody, an isolated antibody hypervariable domain, etc.) which binds specifically with a marker protein or fragment thereof, including a marker protein which has undergone all or a portion of its normal post-translational modification.

In another particular embodiment, expression of a marker is assessed by preparing mRNA/cDNA (i.e., a transcribed polynucleotide) from cells in a subject sample, and by hybridizing the mRNA/cDNA with a reference polynucleotide which is a complement of a marker nucleic acid, or a fragment thereof. cDNA can, optionally, be amplified using any of a variety of polymerase chain reaction methods prior to hybridization with the reference polynucleotide; preferably, it is not amplified. Expression of one or more markers can likewise be detected using quantitative PCR to assess the level of expression of the marker(s). Alternatively, any of the many methods of detecting mutations or variants (e.g., single nucleotide polymorphisms, deletions, etc.) of a marker may be used to detect occurrence of a marker in a subject.

In a related embodiment, a mixture of transcribed polynucleotides obtained from the sample is contacted with a substrate having fixed thereto a polynucleotide complementary to or homologous with at least a portion (e.g., at least 7, 10, 15, 20, 25, 30, 40, 50, 100, 500, or more nucleotide residues) of a marker nucleic acid. If polynucleotides complementary to or homologous with are differentially detectable on the substrate (e.g., detectable using different chromophores or fluorophores, or fixed to different selected positions), then the levels of expression of a plurality of markers can be assessed simultaneously using a single substrate (e.g., a “gene chip” microarray of polynucleotides fixed at selected positions). When a method of assessing marker expression is used which involves hybridization of one nucleic acid with another, it is desired that the hybridization be performed under stringent hybridization conditions.

In certain embodiments, the biomarker assays can be performed using mass spectrometry or surface plasmon resonance. In various embodiments, the method of identifying an agent active against a disorder and/or disease state in a subject can include one or more of: a) providing a sample of cells containing one or more markers or derivative thereof; b) preparing an extract from such cells; c) mixing the extract with a labeled nucleic acid probe containing a marker binding site; and, d) determining the formation of a complex between the marker and the nucleic acid probe in the presence or absence of the test agent. The determining step can include subjecting said extract/nucleic acid probe mixture to an electrophoretic mobility shift assay.

In certain embodiments, the determining step comprises an assay selected from an enzyme linked immunoabsorption assay (ELISA), fluorescence based assays and ultra high throughput assays, for example surface plasmon resonance (SPR) or fluorescence correlation spectroscopy (FCS) assays. In such embodiments, the SPR sensor is useful for direct real-time observation of biomolecular interactions since SPR is sensitive to minute refractive index changes at a metal-dielectric surface. SPR is a surface technique that is sensitive to changes of 10⁵ to 10⁻⁶ refractive index (RI) units within approximately 200 nm of the SPR sensor/sample interface. Thus, SPR spectroscopy is useful for monitoring the growth of thin organic films deposited on the sensing layer.

Because the compositions, kits, and methods rely on detection of a difference in expression levels of one or more markers, it is desired that the level of expression of the marker is significantly greater than the minimum detection limit of the method used to assess expression in at least one of normal cells and colon cancer-affected cells.

It is understood that by routine screening of additional subject samples using one or more of the markers, it will be realized that certain of the markers are over-expressed in cells of various types, including a specific disorder and/or disease state in a subject.

In addition, as a greater number of subject samples are assessed for expression of the markers and the outcomes of the individual subjects from whom the samples were obtained are correlated, it will also be confirmed that altered expression of certain of the markers are strongly correlated with a disorder and/or disease state in a subject and that altered expression of other markers are strongly correlated with other diseases. The compositions, kits, and methods are thus useful for characterizing one or more of the stage, grade, histological type, and nature of a disorder and/or disease state in a subject.

When the compositions, kits, and methods are used for characterizing one or more of the stage, grade, histological type, and nature of a disorder and/or disease state in a subject, it is desired that the marker or panel of markers is selected such that a positive result is obtained in at least about 20%, and in certain embodiments, at least about 40%, 60%, or 80%, and in substantially all subjects afflicted with a disorder and/or disease state of the corresponding stage, grade, histological type, or nature. The marker or panel of markers invention can be selected such that a positive predictive value of greater than about 10% is obtained for the general population (in a non-limiting example, coupled with an assay specificity greater than 80%).

When a plurality of markers are used in the compositions, kits, and methods, the level of expression of each marker in a subject sample can be compared with the normal level of expression of each of the plurality of markers in non-disorder and/or non-disease samples of the same type, either in a single reaction mixture (i.e. using reagents, such as different fluorescent probes, for each marker) or in individual reaction mixtures corresponding to one or more of the markers. In one embodiment, a significantly increased level of expression of more than one of the plurality of markers in the sample, relative to the corresponding normal levels, is an indication that the subject is afflicted with a disorder and/or disease state. When a plurality of markers is used, 2, 3, 4, 5, 8, 10, 12, 15, 20, 30, or 50 or more individual markers can be used; in certain embodiments, the use of fewer markers may be desired.

In order to maximize the sensitivity of the compositions, kits, and methods (i.e. by interference attributable to cells of system origin in a subject sample), it is desirable that the marker used therein be a marker which has a restricted tissue distribution, e.g., normally not expressed in a non-system tissue.

It is recognized that the compositions, kits, and methods will be of particular utility to subjects having an enhanced risk of developing a disorder and/or disease state in a subject and their medical advisors. Subjects recognized as having an enhanced risk of developing a disorder and/or disease include, for example, subjects having a familial history of such disorder or disease.

The level of expression of a marker in normal human system tissue can be assessed in a variety of ways. In one embodiment, this normal level of expression is assessed by assessing the level of expression of the marker in a portion of system cells which appear to be normal and by comparing this normal level of expression with the level of expression in a portion of the system cells which is suspected of being abnormal. Alternately, and particularly as further information becomes available as a result of routine performance of the methods described herein, population-average values for normal expression of the markers may be used. In other embodiments, the ‘normal’ level of expression of a marker may be determined by assessing expression of the marker in a subject sample obtained from a non-afflicted subject, from a subject sample obtained from a subject before the suspected onset of a disorder and/or disease state in the subject, from archived subject samples, and the like.

There is also provided herein compositions, kits, and methods for assessing the presence of disorder and/or disease state cells in a sample (e.g. an archived tissue sample or a sample obtained from a subject). These compositions, kits, and methods are substantially the same as those described above, except that, where necessary, the compositions, kits, and methods are adapted for use with samples other than subject samples. For example, when the sample to be used is a parafinized, archived human tissue sample, it can be necessary to adjust the ratio of compounds in the compositions, in the kits, or the methods used to assess levels of marker expression in the sample.

### Kits and Reagents

The kits are useful for assessing the presence of disease cells (e.g. in a sample such as a subject sample). The kit comprises a plurality of reagents, each of which is capable of binding specifically with a marker nucleic acid or protein. Suitable reagents for binding with a marker protein include antibodies, antibody derivatives, antibody fragments, and the like. Suitable reagents for binding with a marker nucleic acid (e.g. a genomic DNA, an MRNA, a spliced MRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kits may optionally comprise additional components useful for performing the methods described herein. By way of example, the kit may comprise fluids (e.g. SSC buffer) suitable for annealing complementary nucleic acids or for binding an antibody with a protein with which it specifically binds, one or more sample compartments, an instructional material which describes performance of the method, a sample of normal colon system cells, a sample of colon cancer-related disease cells, and the like.

### Methods of Producing Antibodies

There is also provided herein a method of making an isolated hybridoma which produces an antibody useful for assessing whether a subject is afflicted with a disorder and/or disease state. In this method, a protein or peptide comprising the entirety or a segment of a marker protein is synthesized or isolated (e.g. by purification from a cell in which it is expressed or by transcription and translation of a nucleic acid encoding the protein or peptide in vivo or in vitro). A vertebrate, for example, a mammal such as a mouse, rat, rabbit, or sheep, is immunized using the protein or peptide. The vertebrate may optionally (and preferably) be immunized at least one additional time with the protein or peptide, so that the vertebrate exhibits a robust immune response to the protein or peptide. Splenocytes are isolated from the immunized vertebrate and fused with an immortalized cell line to form hybridomas, using any of a variety of methods. Hybridomas formed in this manner are then screened using standard methods to identify one or more hybridomas which produce an antibody which specifically binds with the marker protein or a fragment thereof. There is also provided herein hybridomas made by this method and antibodies made using such hybridomas.

### Methods of Assessing Efficacy

There is also provided herein a method of assessing the efficacy of a test compound for inhibiting disease cells. As described above, differences in the level of expression of the markers correlate with the abnormal state of the subject's cells. Although it is recognized that changes in the levels of expression of certain of the markers likely result from the abnormal state of such cells, it is likewise recognized that changes in the levels of expression of other of the markers induce, maintain, and promote the abnormal state of those cells. Thus, compounds which inhibit a disorder and/or disease state in a subject will cause the level of expression of one or more of the markers to change to a level nearer the normal level of expression for that marker (i.e. the level of expression for the marker in normal cells).

This method thus comprises comparing expression of a marker in a first cell sample and maintained in the presence of the test compound and expression of the marker in a second colon cell sample and maintained in the absence of the test compound. A significantly reduced expression of a marker in the presence of the test compound is an indication that the test compound inhibits a related disease. The cell samples may, for example, be aliquots of a single sample of normal cells obtained from a subject, pooled samples of normal cells obtained from a subject, cells of a normal cell line, aliquots of a single sample of related disease cells obtained from a subject, pooled samples of related disease cells obtained from a subject, cells of a related disease cell line, or the like.

In one embodiment, the samples are cancer-related disease cells obtained from a subject and a plurality of compounds believed to be effective for inhibiting various cancer-related related diseases are tested in order to identify the compound which is likely to best inhibit the cancer-related disease in the subject.

This method may likewise be used to assess the efficacy of a therapy for inhibiting a related disease in a subject. In this method, the level of expression of one or more markers in a pair of samples (one subjected to the therapy, the other not subjected to the therapy) is assessed. As with the method of assessing the efficacy of test compounds, if the therapy induces a significantly lower level of expression of a marker then the therapy is efficacious for inhibiting a cancer-related disease. As above, if samples from a selected subject are used in this method, then alternative therapies can be assessed in vitro in order to select a therapy most likely to be efficacious for inhibiting a cancer-related disease in the subject.

As described herein, the abnormal state of human cells is correlated with changes in the levels of expression of the markers. There is also provided a method for assessing the harmful potential of a test compound. This method comprises maintaining separate aliquots of human cells in the presence and absence of the test compound. Expression of a marker in each of the aliquots is compared. A significantly higher level of expression of a marker in the aliquot maintained in the presence of the test compound (relative to the aliquot maintained in the absence of the test compound) is an indication that the test compound possesses a harmful potential. The relative harmful potential of various test compounds can be assessed by comparing the degree of enhancement or inhibition of the level of expression of the relevant markers, by comparing the number of markers for which the level of expression is enhanced or inhibited, or by comparing both. Various aspects are described in further detail in the following subsections.

### Isolated Proteins and Antibodies

One aspect pertains to isolated marker proteins and biologically active portions thereof, as well as polypeptide fragments suitable for use as immunogens to raise antibodies directed against a marker protein or a fragment thereof. In one embodiment, the native marker protein can be isolated from cells or tissue sources by an appropriate purification scheme using standard protein purification techniques. In another embodiment, a protein or peptide comprising the whole or a segment of the marker protein is produced by recombinant DNA techniques. Alternative to recombinant expression, such protein or peptide can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”).

When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a marker protein include polypeptides comprising amino acid sequences sufficiently identical to or derived from the amino acid sequence of the marker protein, which include fewer amino acids than the full length protein, and exhibit at least one activity of the corresponding full-length protein. Typically, biologically active portions comprise a domain or motif with at least one activity of the corresponding full-length protein. A biologically active portion of a marker protein can be a polypeptide which is, for example, 10, 25, 50, 100 or more amino acids in length. Moreover, other biologically active portions, in which other regions of the marker protein are deleted, can be prepared by recombinant techniques and evaluated for one or more of the functional activities of the native form of the marker protein. In certain embodiments, useful proteins are substantially identical (e.g., at least about 40%, and in certain embodiments, 50%, 60%, 70%, 80%, 90%, 95%, or 99%) to one of these sequences and retain the functional activity of the corresponding naturally-occurring marker protein yet differ in amino acid sequence due to natural allelic variation or mutagenesis.

In addition, libraries of segments of a marker protein can be used to generate a variegated population of polypeptides for screening and subsequent selection of variant marker proteins or segments thereof.

### Predictive Medicine

There is also provided herein uses of the animal models and markers in the field of predictive medicine in which diagnostic assays, prognostic assays, pharmacogenomics, and monitoring clinical trials are used for prognostic (predictive) purposes to thereby treat an individual prophylactically. Accordingly, there is also provided herein diagnostic assays for determining the level of expression of one or more marker proteins or nucleic acids, in order to determine whether an individual is at risk of developing a particular disorder and/or disease. Such assays can be used for prognostic or predictive purposes to thereby prophylactically treat an individual prior to the onset of the disorder and/or disease.

In another aspect, the methods are useful for at least periodic screening of the same individual to see if that individual has been exposed to chemicals or toxins that change his/her expression patterns.

Yet another aspect pertains to monitoring the influence of agents (e.g., drugs or other compounds) administered either to inhibit a disorder and/or disease or to treat or prevent any other disorder (e.g., in order to understand any system effects that such treatment may have) on the expression or activity of a marker in clinical trials.

### Pharmaceutical Compositions

The compounds may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles or microspheres formed of biodegradable or non-biodegradable polymers or proteins or liposomes for targeting to cells. Such systems are well known to those skilled in the art and may be optimized for use with the appropriate nucleic acid.

Various methods for nucleic acid delivery are described, for example in Sambrook et al., 1989, Molecular Cloning: A Laboratory Manual, Cold Spring Harbor Laboratory, New York; and Ausubel et al., 1994, Current Protocols in Molecular Biology, John Wiley & Sons, New York. Such nucleic acid delivery systems comprise the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, with an added preservative. Those of skill in the art can readily determine the various parameters for preparing and formulating the compositions without resort to undue experimentation. The compound can be used alone or in combination with other suitable components.

In general, methods of administering compounds, including nucleic acids, are well known in the art. In particular, the routes of administration already in use for nucleic acid therapeutics, along with formulations in current use, provide preferred routes of administration and formulation for the nucleic acids selected will depend of course, upon factors such as the particular formulation, the severity of the state of the subject being treated, and the dosage required for therapeutic efficacy. As generally used herein, an “effective amount” is that amount which is able to treat one or more symptoms of the disorder, reverse the progression of one or more symptoms of the disorder, halt the progression of one or more symptoms of the disorder, or prevent the occurrence of one or more symptoms of the disorder in a subject to whom the formulation is administered, as compared to a matched subject not receiving the compound. The actual effective amounts of compound can vary according to the specific compound or combination thereof being utilized, the particular composition formulated, the mode of administration, and the age, weight, condition of the individual, and severity of the symptoms or condition being treated.

Any acceptable method known to one of ordinary skill in the art may be used to administer a formulation to the subject. The administration may be localized (i.e., to a particular region, physiological system, tissue, organ, or cell type) or systemic, depending on the condition being treated.

### Pharmacogenomics

The markers are also useful as pharmacogenomic markers. As used herein, a “pharmacogenomic marker” is an objective biochemical marker whose expression level correlates with a specific clinical drug response or susceptibility in a subject. The presence or quantity of the pharmacogenomic marker expression is related to the predicted response of the subject and more particularly the subject's tumor to therapy with a specific drug or class of drugs. By assessing the presence or quantity of the expression of one or more pharmacogenomic markers in a subject, a drug therapy which is most appropriate for the subject, or which is predicted to have a greater degree of success, may be selected.

### Monitoring Clinical Trials

Monitoring the influence of agents (e.g., drug compounds) on the level of expression of a marker can be applied not only in basic drug screening, but also in clinical trials. For example, the effectiveness of an agent to affect marker expression can be monitored in clinical trials of subjects receiving treatment for a colon cancer-related disease.

In one non-limiting embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of:
obtaining a pre-administration sample from a subject prior to administration of the agent;
detecting the level of expression of one or more selected markers in the pre-administration sample;
obtaining one or more post-administration samples from the subject;
detecting the level of expression of the marker(s) in the post-administration samples;
comparing the level of expression of the marker(s) in the pre-administration sample with the level of expression of the marker(s) in the post-administration sample or samples; and
   altering the administration of the agent to the subject accordingly.

For example, increased expression of the marker gene(s) during the course of treatment may indicate ineffective dosage and the desirability of increasing the dosage. Conversely, decreased expression of the marker gene(s) may indicate efficacious treatment and no need to change dosage.

### Electronic Apparatus Readable Media, Systems, Arrays and Methods of Using Same

As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; and general hard disks and hybrids of these categories such as magnetic/optical storage media. The medium is adapted or configured for having recorded thereon a marker as described herein.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as personal digital assistants (PDAs), cellular phone, pager and the like; and local and distributed processing systems.

As used herein, “recorded” refers to a process for storing or encoding information on the electronic apparatus readable medium. Those skilled in the art can readily adopt any method for recording information on media to generate materials comprising the markers described herein.

A variety of software programs and formats can be used to store the marker information of the present invention on the electronic apparatus readable medium. Any number of data processor structuring formats (e.g., text file or database) may be employed in order to obtain or create a medium having recorded thereon the markers. By providing the markers in readable form, one can routinely access the marker sequence information for a variety of purposes. For example, one skilled in the art can use the nucleotide or amino acid sequences in readable form to compare a target sequence or target structural motif with the sequence information stored within the data storage means. Search means are used to identify fragments or regions of the sequences which match a particular target sequence or target motif.

Thus, there is also provided herein a medium for holding instructions for performing a method for determining whether a subject has a cancer-related disease or a pre-disposition to a cancer-related disease, wherein the method comprises the steps of determining the presence or absence of a marker and based on the presence or absence of the marker, determining whether the subject has a cancer-related disease or a pre-disposition to a cancer-related disease and/or recommending a particular treatment for a cancer-related disease or pre-cancer-related disease condition.

There is also provided herein an electronic system and/or in a network, a method for determining whether a subject has a cancer-related disease or a pre-disposition to a cancer-related disease associated with a marker wherein the method comprises the steps of determining the presence or absence of the marker, and based on the presence or absence of the marker, determining whether the subject has a particular disorder and/or disease or a pre-disposition to such disorder and/or disease, and/or recommending a particular treatment for such disease or disease and/or such pre-cancer-related disease condition. The method may further comprise the step of receiving phenotypic information associated with the subject and/or acquiring from a network phenotypic information associated with the subject.

Also provided herein is a network, a method for determining whether a subject has a disorder and/or disease or a pre-disposition to a disorder and/or disease associated with a marker, the method comprising the steps of receiving information associated with the marker, receiving phenotypic information associated with the subject, acquiring information from the network corresponding to the marker and/or disorder and/or disease, and based on one or more of the phenotypic information, the marker, and the acquired information, determining whether the subject has a disorder and/or disease or a pre-disposition thereto. The method may further comprise the step of recommending a particular treatment for the disorder and/or disease or pre-disposition thereto.

There is also provided herein a business method for determining whether a subject has a disorder and/or disease or a pre-disposition thereto, the method comprising the steps of receiving information associated with the marker, receiving phenotypic information associated with the subject, acquiring information from the network corresponding to the marker and/or a disorder and/or disease, and based on one or more of the phenotypic information, the marker, and the acquired information, determining whether the subject has a disorder and/or disease or a pre-disposition thereto. The method may further comprise the step of recommending a particular treatment therefor.

There is also provided herein an array that can be used to assay expression of one or more genes in the array. In one embodiment, the array can be used to assay gene expression in a tissue to ascertain tissue specificity of genes in the array. In this manner, up to about 7000 or more genes can be simultaneously assayed for expression. This allows a profile to be developed showing a battery of genes specifically expressed in one or more tissues.

In addition to such qualitative determination, there is provided herein the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined.

Such a determination is useful, for example, to know the effect of cell-cell interaction at the level of gene expression. If an agent is administered therapeutically to treat one cell type but has an undesirable effect on another cell type, the method provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a disorder and/or disease, progression thereof, and processes, such as cellular transformation associated therewith.

The array is also useful for ascertaining the effect of the expression of a gene or the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention.

### Surrogate Markers

The markers may serve as surrogate markers for one or more disorders or disease states or for conditions leading up thereto. As used herein, a “surrogate marker” is an objective biochemical marker which correlates with the absence or presence of a disease or disorder, or with the progression of a disease or disorder. The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies, or when an assessment of disease progression is desired before a potentially dangerous clinical endpoint is reached.

The markers are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo.

Pharmacodynamic markers are of particular use in increasing the sensitivity of detection of drug effects, particularly when the drug is administered in low doses. Since even a small amount of a drug may be sufficient to activate multiple rounds of marker transcription or expression, the amplified marker may be in a quantity which is more readily detectable than the drug itself. Also, the marker may be more easily detected due to the nature of the marker itself; for example, using the methods described herein, antibodies may be employed in an immune-based detection system for a protein marker, or marker-specific radiolabeled probes may be used to detect a mRNA marker. Furthermore, the use of a pharmacodynamic marker may offer mechanism-based prediction of risk due to drug treatment beyond the range of possible direct observations.

### Protocols for Testing

The method of testing for a disorder and/or disease may comprise, for example measuring the expression level of each marker gene in a biological sample from a subject over time and comparing the level with that of the marker gene in a control biological sample.

When the marker gene is one of the genes described herein and the expression level is differentially expressed (for examples, higher or lower than that in the control), the subject is judged to be affected with a disorder and/or disease. When the expression level of the marker gene falls within the permissible range, the subject is unlikely to be affected therewith.

The standard value for the control may be pre-determined by measuring the expression level of the marker gene in the control, in order to compare the expression levels. For example, the standard value can be determined based on the expression level of the above-mentioned marker gene in the control. For example, in certain embodiments, the permissible range is taken as ±2S.D. based on the standard value. Once the standard value is determined, the testing method may be performed by measuring only the expression level in a biological sample from a subject and comparing the value with the determined standard value for the control.

Expression levels of marker genes include transcription of the marker genes to mRNA, and translation into proteins. Therefore, one method of testing for a disorder and/or disease is performed based on a comparison of the intensity of expression of mRNA corresponding to the marker genes, or the expression level of proteins encoded by the marker genes.

The measurement of the expression levels of marker genes in the testing for a disorder and/or disease can be carried out according to various gene analysis methods. Specifically, one can use, for example, a hybridization technique using nucleic acids that hybridize to these genes as probes, or a gene amplification technique using DNA that hybridize to the marker genes as primers.

The probes or primers used for the testing can be designed based on the nucleotide sequences of the marker genes. The identification numbers for the nucleotide sequences of the respective marker genes are described herein.

Further, it is to be understood that genes of higher animals generally accompany polymorphism in a high frequency. There are also many molecules that produce isoforms comprising mutually different amino acid sequences during the splicing process. Any gene associated with a colon cancer-related disease that has an activity similar to that of a marker gene is included in the marker genes, even if it has nucleotide sequence differences due to polymorphism or being an isoform.

It is also to be understood that the marker genes can include homologs of other species in addition to humans. Thus, unless otherwise specified, the expression “marker gene” refers to a homolog of the marker gene unique to the species or a foreign marker gene which has been introduced into an individual.

Also, it is to be understood that a “homolog of a marker gene” refers to a gene derived from a species other than a human, which can hybridize to the human marker gene as a probe under stringent conditions. Such stringent conditions are known to one skilled in the art who can select an appropriate condition to produce an equal stringency experimentally or empirically.

A polynucleotide comprising the nucleotide sequence of a marker gene or a nucleotide sequence that is complementary to the complementary strand of the nucleotide sequence of a marker gene and has at least 15 nucleotides, can be used as a primer or probe. Thus, a “complementary strand” means one strand of a double stranded DNA with respect to the other strand and which is composed of A:T (U for RNA) and G:C base pairs.

In addition, “complementary” means not only those that are completely complementary to a region of at least 15 continuous nucleotides, but also those that have a nucleotide sequence homology of at least 40% in certain instances, 50% in certain instances, 60% in certain instances, 70% in certain instances, 80% in certain instances, 90% in certain instances, and 95% in certain instances, or higher. The degree of homology between nucleotide sequences can be determined by an algorithm, BLAST, etc.

Such polynucleotides are useful as a probe to detect a marker gene, or as a primer to amplify a marker gene. When used as a primer, the polynucleotide comprises usually 15 by to 100 bp, and in certain embodiments 15 by to 35 by of nucleotides. When used as a probe, a DNA comprises the whole nucleotide sequence of the marker gene (or the complementary strand thereof), or a partial sequence thereof that has at least 15 by nucleotides. When used as a primer, the 3′ region must be complementary to the marker gene, while the 5′ region can be linked to a restriction enzyme-recognition sequence or a tag.

“Polynucleotides” may be either DNA or RNA. These polynucleotides may be either synthetic or naturally-occurring. Also, DNA used as a probe for hybridization is usually labeled. Those skilled in the art readily understand such labeling methods. Herein, the term “oligonucleotide” means a polynucleotide with a relatively low degree of polymerization. Oligonucleotides are included in polynucleotides.

Tests for a disorder and/or disease using hybridization techniques can be performed using, for example, Northern hybridization, dot blot hybridization, or the DNA microarray technique. Furthermore, gene amplification techniques, such as the RT-PCR method may be used. By using the PCR amplification monitoring method during the gene amplification step in RT-PCR, one can achieve a more quantitative analysis of the expression of a marker gene.

In the PCR gene amplification monitoring method, the detection target (DNA or reverse transcript of RNA) is hybridized to probes that are labeled with a fluorescent dye and a quencher which absorbs the fluorescence. When the PCR proceeds and Taq polymerase degrades the probe with its 5′-3′ exonuclease activity, the fluorescent dye and the quencher draw away from each other and the fluorescence is detected. The fluorescence is detected in real time. By simultaneously measuring a standard sample in which the copy number of a target is known, it is possible to determine the copy number of the target in the subject sample with the cycle number where PCR amplification is linear. Also, one skilled in the art recognizes that the PCR amplification monitoring method can be carried out using any suitable method.

The method of testing for a colon cancer-related disease can be also carried out by detecting a protein encoded by a marker gene. Hereinafter, a protein encoded by a marker gene is described as a “marker protein.” For such test methods, for example, the Western blotting method, the immunoprecipitation method, and the ELISA method may be employed using an antibody that binds to each marker protein.

Antibodies used in the detection that bind to the marker protein may be produced by any suitable technique. Also, in order to detect a marker protein, such an antibody may be appropriately labeled. Alternatively, instead of labeling the antibody, a substance that specifically binds to the antibody, for example, protein A or protein G, may be labeled to detect the marker protein indirectly. More specifically, such a detection method can include the ELISA method.

A protein or a partial peptide thereof used as an antigen may be obtained, for example, by inserting a marker gene or a portion thereof into an expression vector, introducing the construct into an appropriate host cell to produce a transformant, culturing the transformant to express the recombinant protein, and purifying the expressed recombinant protein from the culture or the culture supernatant. Alternatively, the amino acid sequence encoded by a gene or an oligopeptide comprising a portion of the amino acid sequence encoded by a full-length cDNA are chemically synthesized to be used as an immunogen.

Furthermore, a test for a colon cancer-related disease can be performed using as an index not only the expression level of a marker gene but also the activity of a marker protein in a biological sample. Activity of a marker protein means the biological activity intrinsic to the protein. Various methods can be used for measuring the activity of each protein.

Even if a subject is not diagnosed as being affected with a disorder and/or disease in a routine test in spite of symptoms suggesting these diseases, whether or not such a subject is suffering from a disorder and/or disease can be easily determined by performing a test according to the methods described herein.

More specifically, in certain embodiments, when the marker gene is one of the genes described herein, an increase or decrease in the expression level of the marker gene in a subject whose symptoms suggest at least a susceptibility to a disorder and/or disease indicates that the symptoms are primarily caused thereby.

In addition, the tests are useful to determine whether a disorder and/or disease is improving in a subject. In other words, the methods described herein can be used to judge the therapeutic effect of a treatment therefor. Furthermore, when the marker gene is one of the genes described herein, an increase or decrease in the expression level of the marker gene in a subject, who has been diagnosed as being affected thereby, implies that the disease has progressed more.

The severity and/or susceptibility to a disorder and/or disease may also be determined based on the difference in expression levels. For example, when the marker gene is one of the genes described herein, the degree of increase in the expression level of the marker gene is correlated with the presence and/or severity of a disorder and/or disease.

### Animal Models

Animal models for a disorder and/or disease where the expression level of one or more marker genes or a gene functionally equivalent to the marker gene has been elevated in the animal model can also be made. A “functionally equivalent gene” as used herein generally is a gene that encodes a protein having an activity similar to a known activity of a protein encoded by the marker gene. A representative example of a functionally equivalent gene includes a counterpart of a marker gene of a subject animal, which is intrinsic to the animal.

The animal model is useful for detecting physiological changes due to a disorder and/or disease. In certain embodiments, the animal model is useful to reveal additional functions of marker genes and to evaluate drugs whose targets are the marker genes.

An animal model can be created by controlling the expression level of a counterpart gene or administering a counterpart gene. The method can include creating an animal model by controlling the expression level of a gene selected from the group of genes described herein. In another embodiment, the method can include creating an animal model by administering the protein encoded by a gene described herein, or administering an antibody against the protein. It is to be also understood, that in certain other embodiments, the marker can be over-expressed such that the marker can then be measured using appropriate methods. In another embodiment, an animal model can be created by introducing a gene selected from such groups of genes, or by administering a protein encoded by such a gene. In another embodiment, a disorder and/or disease can be induced by suppressing the expression of a gene selected from such groups of genes or the activity of a protein encoded by such a gene. An antisense nucleic acid, a ribozyme, or an RNAi can be used to suppress the expression. The activity of a protein can be controlled effectively by administering a substance that inhibits the activity, such as an antibody.

The animal model is useful to elucidate the mechanism underlying a disorder and/or disease and also to test the safety of compounds obtained by screening. For example, when an animal model develops the symptoms of a particular disorder and/or disease, or when a measured value involved in a certain disorder and/or disease alters in the animal, a screening system can be constructed to explore compounds having activity to alleviate the disease.

As used herein, the expression “an increase in the expression level” refers to any one of the following: where a marker gene introduced as a foreign gene is expressed artificially; where the transcription of a marker gene intrinsic to the subject animal and the translation thereof into the protein are enhanced; or where the hydrolysis of the protein, which is the translation product, is suppressed.

As used herein, the expression “a decrease in the expression level” refers to either the state in which the transcription of a marker gene of the subject animal and the translation thereof into the protein are inhibited, or the state in which the hydrolysis of the protein, which is the translation product, is enhanced. The expression level of a gene can be determined, for example, by a difference in signal intensity on a DNA chip. Furthermore, the activity of the translation product—the protein—can be determined by comparing with that in the normal state.

It is also within the contemplated scope that the animal model can include transgenic animals, including, for example animals where a marker gene has been introduced and expressed artificially; marker gene knockout animals; and knock-in animals in which another gene has been substituted for a marker gene. A transgenic animal, into which an antisense nucleic acid of a marker gene, a ribozyme, a polynucleotide having an RNAi effect, or a DNA functioning as a decoy nucleic acid or such has been introduced, can be used as the transgenic animal. Such transgenic animals also include, for example, animals in which the activity of a marker protein has been enhanced or suppressed by introducing a mutation(s) into the coding region of the gene, or the amino acid sequence has been modified to become resistant or susceptible to hydrolysis. Mutations in an amino acid sequence include substitutions, deletions, insertions, and additions.

### Examples of Expression

In addition, the expression itself of a marker gene can be controlled by introducing a mutation(s) into the transcriptional regulatory region of the gene. Those skilled in the art understand such amino acid substitutions. Also, the number of amino acids that are mutated is not particularly restricted, as long as the activity is maintained. Normally, it is within 50 amino acids, in certain non-limiting embodiments, within 30 amino acids, within 10 amino acids, or within 3 amino acids. The site of mutation may be any site, as long as the activity is maintained.

In yet another aspect, there is provided herein screening methods for candidate compounds for therapeutic agents to treat a particular disorder and/or disease. One or more marker genes are selected from the group of genes described herein. A therapeutic agent for a colon cancer-related disease can be obtained by selecting a compound capable of increasing or decreasing the expression level of the marker gene(s).

It is to be understood that the expression “a compound that increases the expression level of a gene” refers to a compound that promotes any one of the steps of gene transcription, gene translation, or expression of a protein activity. On the other hand, the expression “a compound that decreases the expression level of a gene”, as used herein, refers to a compound that inhibits any one of these steps.

In particular aspects, the method of screening for a therapeutic agent for a disorder and/or disease can be carried out either in vivo or in vitro. This screening method can be performed, for example, by:
administering a candidate compound to an animal subject;
measuring the expression level of a marker gene(s) in a biological sample from the animal subject; or
selecting a compound that increases or decreases the expression level of a marker gene(s) as compared to that in a control with which the candidate compound has not been contacted.

In still another aspect, there is provided herein a method to assess the efficacy of a candidate compound for a pharmaceutical agent on the expression level of a marker gene(s) by contacting an animal subject with the candidate compound and monitoring the effect of the compound on the expression level of the marker gene(s) in a biological sample derived from the animal subject. The variation in the expression level of the marker gene(s) in a biological sample derived from the animal subject can be monitored using the same technique as used in the testing method described above. Furthermore, based on the evaluation, a candidate compound for a pharmaceutical agent can be selected by screening.

All patents, patent applications and references cited herein are incorporated in their entirety by reference. While the invention has been described and exemplified in sufficient detail for those skilled in this art to make and use it, various alternatives, modifications and improvements should be apparent without departing from the spirit and scope of the invention. One skilled in the art readily appreciates that the present invention is well adapted to carry out the objects and obtain the ends and advantages mentioned, as well as those inherent therein.

### Certain Nucleobase Sequences

Nucleobase sequences of mature miRNAs and their corresponding stem-loop sequences described herein are the sequences found in miRBase, an online searchable database of miRNA sequences and annotation, found athttp://microrna.sanger.ac.uk/. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compounds that may encompass such modified oligonucleotides may be complementary to any nucleobase sequence version of the miRNAs described herein.

It is understood that any nucleobase sequence set forth herein is independent of any modification to a sugar moiety, an internucleoside linkage, or a nucleobase. It is further understood that a nucleobase sequence comprising U's also encompasses the same nucleobase sequence wherein ‘U’ is replaced by ‘T’ at one or more positions having ‘U’. Conversely, it is understood that a nucleobase sequence comprising T's also encompasses the same nucleobase sequence wherein ‘T’ is replaced by ‘U’ at one or more positions having ‘T’.

In certain embodiments, a modified oligonucleotide has a nucleobase sequence that is complementary to a miRNA or a precursor thereof, meaning that the nucleobase sequence of a modified oligonucleotide is a least 60%, 65%, 70%, 75%, 80%, 85%, 90%, 95%, 97%, 98% or 99% identical to the complement of a miRNA or precursor thereof over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the two sequences hybridize under stringent hybridization conditions. Accordingly, in certain embodiments the nucleobase sequence of a modified oligonucleotide may have one or more mismatched basepairs with respect to its target miRNA or target miRNA precursor sequence, and is capable of hybridizing to its target sequence. In certain embodiments, a modified oligonucleotide has a nucleobase sequence that is 100% complementary to a miRNA or a precursor thereof. In certain embodiments, the nucleobase sequence of a modified oligonucleotide has full-length complementary to a miRNA.

miRNA (miR) Therapies

In some embodiments, the present invention provides microRNAs that inhibit the expression of one or more genes in a subject. MicroRNA expression profiles can serve as a new class of cancer biomarkers.

Included herein are methods of inhibiting gene expression and/or activity using one or more MiRs. In some embodiments, the miR(s) inhibit the expression of a protein. In other embodiments, the miRNA(s) inhibits gene activity (e.g., cell invasion activity).

The miRNA can be isolated from cells or tissues, recombinantly produced, or synthesized in vitro by a variety of techniques well known to one of ordinary skill in the art. In one embodiment, miRNA is isolated from cells or tissues. Techniques for isolating miRNA from cells or tissues are well known to one of ordinary skill in the art. For example, miRNA can be isolated from total RNA using the mirVana miRNA isolation kit from Ambion, Inc. Another technique utilizes the flashIPAGE™ Fractionator System (Ambion, Inc.) for PAGE purification of small nucleic acids.

For the use of miRNA therapeutics, it is understood by one of ordinary skill in the art that nucleic acids administered in vivo are taken up and distributed to cells and tissues.

The nucleic acid may be delivered in a suitable manner which enables tissue-specific uptake of the agent and/or nucleic acid delivery system. The formulations described herein can supplement treatment conditions by any known conventional therapy, including, but not limited to, antibody administration, vaccine administration, administration of cytotoxic agents, natural amino acid polypeptides, nucleic acids, nucleotide analogues, and biologic response modifiers. Two or more combined compounds may be used together or sequentially.

Certain embodiments of the invention provide pharmaceutical compositions containing (a) one or more nucleic acid or small molecule compounds and (b) one or more other chemotherapeutic agents.

### Additional Useful Definitions

“Subject” means a human or non-human animal selected for treatment or therapy. “Subject suspected of having” means a subject exhibiting one or more clinical indicators of a disorder, disease or condition.

“Preventing” or “prevention” refers to delaying or forestalling the onset, development or progression of a condition or disease for a period of time, including weeks, months, or years. “Treatment” or “treat” means the application of one or more specific procedures used for the cure or amelioration of a disorder and/or disease. In certain embodiments, the specific procedure is the administration of one or more pharmaceutical agents.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Subject in need thereof” means a subject identified as in need of a therapy or treatment.

“Administering” means providing a pharmaceutical agent or composition to a subject, and includes, but is not limited to, administering by a medical professional and self-administering.

“Parenteral administration” means administration through injection or infusion. Parenteral administration includes, but is not limited to, subcutaneous administration, intravenous administration, intramuscular administration, intraarterial administration, and intracranial administration. “Subcutaneous administration” means administration just below the skin.

“Improves function” means the changes function toward normal parameters. In certain embodiments, function is assessed by measuring molecules found in a subject's bodily fluids. “Pharmaceutical composition” means a mixture of substances suitable for administering to an individual that includes a pharmaceutical agent. For example, a pharmaceutical composition may comprise a modified oligonucleotide and a sterile aqueous solution.

“Target nucleic acid,” “target RNA,” “target RNA transcript” and “nucleic acid target” all mean a nucleic acid capable of being targeted by antisense compounds. “Targeting” means the process of design and selection of nucleobase sequence that will hybridize to a target nucleic acid and induce a desired effect. “Targeted to” means having a nucleobase sequence that will allow hybridization to a target nucleic acid to induce a desired effect. In certain embodiments, a desired effect is reduction of a target nucleic acid.

“Modulation” means to a perturbation of function or activity. In certain embodiments, modulation means an increase in gene expression. In certain embodiments, modulation means a decrease in gene expression.

“Expression” means any functions and steps by which a gene's coded information is converted into structures present and operating in a cell.

“Region” means a portion of linked nucleosides within a nucleic acid. In certain embodiments, a modified oligonucleotide has a nucleobase sequence that is complementary to a region of a target nucleic acid. For example, in certain such embodiments a modified oligonucleotide is complementary to a region of a miRNA stem-loop sequence. In certain such embodiments, a modified oligonucleotide is 100% identical to a region of a miRNA sequence.

“Segment” means a smaller or sub-portion of a region.

“Nucleobase sequence” means the order of contiguous nucleobases, in a 5′ to 3′ orientation, independent of any sugar, linkage, and/or nucleobase modification.

“Contiguous nucleobases” means nucleobases immediately adjacent to each other in a nucleic acid.

“Nucleobase complementarity” means the ability of two nucleobases to pair non-covalently via hydrogen bonding. “Complementary” means a first nucleobase sequence is at least 60%, 65%, 70%, 75%, 80%, 85%, 90%, 95%, 97%, 98% or 99% identical, or is 100% identical, to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the two sequences hybridize under stringent hybridization conditions. In certain embodiments a modified oligonucleotide that has a nucleobase sequence which is 100% complementary to a miRNA, or precursor thereof, may not be 100% complementary to the miRNA, or precursor thereof, over the entire length of the modified oligonucleotide.

“Complementarity” means the nucleobase pairing ability between a first nucleic acid and a second nucleic acid. “Full-length complementarity” means each nucleobase of a first nucleic acid is capable of pairing with each nucleobase at a corresponding position in a second nucleic acid. For example, in certain embodiments, a modified oligonucleotide can mean where each nucleobase has complementarity to a nucleobase in an miRNA has full-length complementarity to the miRNA.

“Percent complementary” means the number of complementary nucleobases in a nucleic acid divided by the length of the nucleic acid. In certain embodiments, percent complementarity of a modified oligonucleotide means the number of nucleobases that are complementary to the target nucleic acid, divided by the number of nucleobases of the modified oligonucleotide. In certain embodiments, percent complementarity of a modified oligonucleotide means the number of nucleobases that are complementary to a miRNA, divided by the number of nucleobases of the modified oligonucleotide.

“Percent region bound” means the percent of a region complementary to an oligonucleotide region. Percent region bound is calculated by dividing the number of nucleobases of the target region that are complementary to the oligonucleotide by the length of the target region. In certain embodiments, percent region bound is at least 80%, at least 85%, at least 90%, at least 95%, at least 96%, at least 97%, at least 98%, at least 99%, or 100%.

“Percent identity” means the number of nucleobases in first nucleic acid that are identical to nucleobases at corresponding positions in a second nucleic acid, divided by the total number of nucleobases in the first nucleic acid.

“Substantially identical” used herein may mean that a first and second nucleobase sequence are at least 60%, 65%, 70%, 75%, 80%, 85%, 90%, 95%, 97%, 98% or 99% identical, or 100% identical, over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases.

“Hybridize” means the annealing of complementary nucleic acids that occurs through nucleobase complementarity.

“Mismatch” means a nucleobase of a first nucleic acid that is not capable of pairing with a nucleobase at a corresponding position of a second nucleic acid.

“Non-complementary nucleobase” means two nucleobases that are not capable of pairing through hydrogen bonding.

“Identical” means having the same nucleobase sequence.

“miRNA” or “miR” means a non-coding RNA between 18 and 25 nucleobases in length which hybridizes to and regulates the expression of a coding RNA. In certain embodiments, a miRNA is the product of cleavage of a pre-miRNA by the enzyme Dicer. Examples of miRNAs are found in the miRNA database known as miRBase (http://microrna.sanger.ac.uk/).

“Pre-miRNA” or “pre-miR” means a non-coding RNA having a hairpin structure, which contains a miRNA. In certain embodiments, a pre-miRNA is the product of cleavage of a pri-miR by the double-stranded RNA-specific ribonuclease known as Drosha.

“Stem-loop sequence” means an RNA having a hairpin structure and containing a mature miRNA sequence. Pre-miRNA sequences and stem-loop sequences may overlap. Examples of stem-loop sequences are found in the miRNA database known as miRBase (microrna.sanger.ac.uk).

“miRNA precursor” means a transcript that originates from a genomic DNA and that comprises a non-coding, structured RNA comprising one or more miRNA sequences. For example, in certain embodiments a miRNA precursor is a pre-miRNA. In certain embodiments, a miRNA precursor is a pri-miRNA.

“Antisense compound” means a compound having a nucleobase sequence that will allow hybridization to a target nucleic acid. In certain embodiments, an antisense compound is an oligonucleotide having a nucleobase sequence complementary to a target nucleic acid.

“Oligonucleotide” means a polymer of linked nucleosides, each of which can be modified or unmodified, independent from one another. “Naturally occurring internucleoside linkage” means a 3′ to 5′ phosphodiester linkage between nucleosides. “Natural nucleobase” means a nucleobase that is unmodified relative to its naturally occurring form. “miR antagonist” means an agent designed to interfere with or inhibit the activity of a miRNA. In certain embodiments, a miR antagonist comprises an antisense compound targeted to a miRNA. In certain embodiments, a miR antagonist comprises a modified oligonucleotide having a nucleobase sequence that is complementary to the nucleobase sequence of a miRNA, or a precursor thereof. In certain embodiments, an miR antagonist comprises a small molecule, or the like that interferes with or inhibits the activity of an miRNA.

The methods and reagents described herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims. It will also be readily apparent to a person skilled in the art that varying substitutions and modifications may be made to the invention disclosed herein without departing from the scope and spirit of the invention.

It should be understood that although the present invention has been specifically disclosed by preferred embodiments and optional features, modifications and variations of the concepts herein disclosed may be resorted to by those skilled in the art, and that such modifications and variations are considered to be within the scope of this invention as defined by the appended claims.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

### REFERENCES

The publication and other material used herein to illuminate the invention or provide additional details respecting the practice of the invention, are incorporated by reference herein, and for convenience are provided in the following bibliography.

Citation of any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

### REFERENCES FOR EXAMPLE I

1. Lowenberg B, Downing J R, Burnett A. Acute myeloid leukemia. N Engl J Med 1999; 341:1051-1062.
2. Burnett A K. Current controversies: which patients with acute myeloid leukemia should receive bone marrow transplantation? An adult theater's view. Br. J Haematol 2002; 118:357-364.
3. Drobyski W R. The role of allogeneic transplantation in high-risk acute myeloid leukemia. Leukemia 2004; 10:1565-1568.
4. Bartel D. MicroRNAs: genomics, biogenesis, mechanism, and function. Cell 2004; 116:281-297.
5. Chen CZ, Li L, Lodish H, Bartel D. MicroRNAs modulate hematopoietic lineage differentiation. Science 2004; 303:83-86.
6. Fazi F, Rosa A, Fatica A, et al. A mini-circuitry comprising microRNA-223 and transcription factors NFI-A and C/EBPa regulates human granulopoiesis. Cell 2005; 123:819-831.
7. Felli N, Fontana L, Pelosi L, et al. MicroRNAs 221 and 222 inhibit normal erythropoiesis and erythroleukemic cell growth via kit receptor down-modulation. Proc Natl Acad Sci USA 2005; 102:18081-18086.
8. Garzon R, Pichiorri F, Palumbo T, et al. MicroRNAs fingerprints during human megakaryocytopoiesis. Proc Natl Acad Sci USA 2006; 103:5078-5083.
9. Calin G A, Dumitru C D, Shimizu M, et al. Frequent deletions and down-regulation of micro-RNA genes miR-15 and miR-16 at 13q14 in chronic lymphocytic leukemia. Proc Natl Acad Sci USA 2002; 99:15524-15529.
10. Volinia S, Calin G, Liu C G, et al. A microRNA expression signature in human solid tumors defines cancer targets. Proc Natl Acad Sci USA 2006; 103:2257-2261.
11. Lu J, Getz G, Miska E A, et al. MicroRNA expression profiles classify human cancers. Nature 2005; 435:834-838.
12. Kornblau S M, Womble M, Cade J S, et al. Comparative analysis of the effects of sample source and test methodology on the assessment of protein expression in acute myelogenous leukemia. Leukemia 2005; 19:1550-1557.
13. ISCN 2005: An International System for Human Cytogenetic Nomenclature. 2005; In Shaffer L G and Tommerup N (Eds.). Basel, Switzerland S. Karger.
14. Kiyoi H, Naoe T, Nakano Y, et al. Prognostic implication of FLT3 and N-RAS gene mutations in acute myeloid leukemia. Blood 1999; 93:3074-3080.
15. Liu C G, Calin G A, Meloon B, et al. An oligonucleotide microchip for genomic-wide microRNA profiling in human and mouse tissues. Proc Natl Acad Sci USA 2004; 11755-11760.
16. Chen C, Ridzon D A, Broomer A J, et al. Real-time quantification of microRNAs by stem-loop RT-PCR. Nucleic Acids Res 2005; 33:e179.
17. Livak K J and Schmittgen T D. Analysis of relative gene expression data using real-time quantitative PCR and the 2(-Delta Delta C (T)) method. Methods 2001; 25:402-408
18. Tusher V G, Tibshirani R, Chu G. Significant analysis of microarrays applied to the ionizing radiation response. Proc Natl Acad Sci USA 2001; 98:5116-5121.
19. World Health Organization Classifications of Tumors. Pathology and Genetics of Tumors of Haematopoietic and Lymph Tissues. 2001; In Jaffe E S, Harris N L, Stein H, Vardinam J W (Eds.). Lyon, France IARC.
20. He L, He X, Lim L P, et al. A microRNA component of the p53 tumour suppressor network. Nature 2007; 447:1130-1134.
21. Cimmino A, Calin G A, Fabbri M, et al. MiR-15 and miR-16 induce apoptosis by targeting BCL2. Proc Natl Acad Sci USA 2005; 102:13944-13949.
22. Johnson S M, Grosshans H, Shingara J, et al. RAS is regulated by the let-7 microRNA family. Cell 120:635-647.
23. Mott J L, Kobayashi S, Bronk S F, Gores G J. mir-29 regulates Mc1-1 protein expression and apoptosis. Oncogene 2007; 26:6133-6140.
24. Pekarsky Y, Santaman U, Cimmino A, et al. Tc11 expression in chronic lymphocytic leukemia is regulated by miR-29 and miR-181. Cancer Res 2006; 66:11590-11593.
25. Voorhoeve P M, Le Sage C, Schrier M, et al. A genetic screen implicates miRNA-372 and miRNA-373 as oncogenes in testicular germ cell tumors. Cell 2006; 124:1169-1181.
26. Yekta S, Shih I H, Bartel D P. MicroRNA-directed cleavage of HOXB8 mRNA. Science 2004; 304:594-596.
27. Meng F, Henson R, Lang M, et al. Involvement of human micro-RNA in growth and response to chemotherapy in human cholangiocarcinoma cell lines. Gastroenterology 2006; 130:2113-2129.
28. Lewis B, Shih I, Jones-Rhoades M, Bartel D, Burge C. Prediction of mammalian microRNA targets. Cell 2003;787-798.
29. Ryu B and Kern S E. The essential similarity of TGFbeta and activin receptor transcriptional responses in cancer cells. Cancer Biol Ther 2003; 2:164-170.
30. Lim L P, Lau L C, Garrett-Engele P, et al. Microarray analysis shows that some microRNAs down-regulate large numbers of target mRNAs. Nature 2005; 433:769-773.
31. Zhou P, Qian L, Kozopas K M, et al. Mc1-1, a Bc1-2 family member, delays the death of hematopoietic cells under a variety of apoptosis-inducing conditions. Blood 1997; 89:630.
32. Stirewalt D L, Kopecky K J, Meshinchi S, et al. FLT3, RAS, and TP53 mutations in elderly patients with acute myeloid leukemia. Blood 2001; 97:3589-3595.
33. Felli N, Pelosi E, Beta R, et al. Lineage-specific expression and functional relevance of microRNA genes in normal hematopoiesis [abstract]. Blood 2006; 106:11a Abstract 2263.
34. Si M L, Zhu S, Wu H, Lu Z, Wu F, Mo Y Y. MiR-21-mediated tumor growth Oncogene 2007; 26:2799-2803.
35. Byrd J C, Mrózek K, Dodge R K. Pretreatment cytogenetic abnormalities are predictive of induction success, cumulative incidence of relapse, and overall survival in adult patients with de novo acute myeloid leukemia: results from Cancer and Leukemia Group B (CALGB 8461). Blood 2002; 100:4325-4336.
36. Calin G A, Ferracin M, Cimmino A, et al. A MicroRNA signature associated with prognosis and progression in chronic lymphocytic leukemia. N Engl J Med 2005; 353:1793-1801.
37. Yanaihara N, Caplen N, Bowman E, et al. Unique microRNA molecular profiles in lung cancer diagnosis and prognosis. Cancer Cell 2006; 9:189-198.
38. Georgantas G, Hildreth R, Morisot S, et al. CD34+ hematopoietic stem-cell progenitor cell microRNA expression and function: a circuit diagram of differentiation control. Proc Natl Acad Sci USA 2007; 104:2750-2755.
39. Costinean S, Zanesi N, Pekarsky Y, et al. Pre-B cell proliferation and lymphoblastic leukemia/high-grade lymphoma in Eμ-miR155 transgenic mice. Proc Natl Acad Sci USA 2006; 103:7024-7029.

### REFERENCES FOR EXAMPLE II

1. Griffiths-Jones S. The microRNA registry. Nucleic Acids Res. 2004; 32, Database issue D109-D111.
2. Tusher V G, Tibshirani R, Chu G. Significant analysis of microarrays applied to the ionizing radiation response. Proc Natl Acad Sci USA. 2001; 98:5116:21.
3. Livak K J, Schmittgen T D. Analysis of relative gene expression data using real-time quantitative PCR and the 2(-Delta Delta C (T)) method. Methods. 2001; 25:402-408.

1. A miRNA signature for predicting outcome of a patient suffering from acute myeloid leukemia (AML), independently from other factors, comprising: a distinct signature of miRNA expression compared with normal CD34⁺ progenitor cells,
wherein the signature comprises one or more of down-regulated miRNAs and none up-regulated in AML samples compared with CD34⁺ normal cells selected from the miRs: hsa-miR-126, hsa-miR-130a, hsa-miR-135, hsa-miR-93, hsa-miR-146, hsa-miR-106b, hsa-miR-224, hsa-miR-125a, hsa-miR-92, hsa-miR-106a, hsa-miR-95, hsa-miR-155, hsa-miR-25, hsa-miR-96, hsa-miR-124a, hsa-miR-18, hsa-miR-20, hsa-let-7d, hsa-miR-26a, hsa-miR-222, hsa-miR-101, hsa-miR-338, hsa-miR-371, hsa-miR-199b, hsa-miR-29b, and hsa-miR-301.

2. A miRNA signature of claim 1, wherein the miRs comprise one or more of: miR-126, miR-130a, miR-93, miR-146, miR-106b, and miR-125a.

3. (canceled)

4. A miRNA signature for determining diagnosing whether a subject has or will develop AML comprising
examining a sample from the subject, and
determining whether there is a positive correlation of expression of miRs with high white blood count: the miRs comprising one or more of: hsa-miR-155, hsa-miR-30e, hsa-miR-23b, hsa-miR-181b, hsa-miR-221, hsa-miR-29b, hsa-miR-95, hsa-miR-128b, hsa-miR-27a, hsa-miR-181c, hsa-miR-921, hsa-miR-181a, hsa-miR-23a, hsa-miR-214, hsa-miR-30b, hsa-miR-30c, hsa-miR-26b, hsa-miR-21, and hsa-miR-222.

5. A miRNA signature for determining whether a subject has or will develop AML comprising examining a sample from the subject and determining whether there is a positive correlation of expression of miRs with high bone marrow (BM) blasts, the miRs comprising one or more of: hsa-miR-30b, hsa-miR-30c, hsa-miR-192, hsa-miR-181a, hsa-miR-155, hsa-let-7a-2, hsa-miR-181b, hsa-miR-181c, hsa-miR-219, hsa-miR-214, and hsa-miR-26a.

6. A miRNA signature for determining whether a subject has or will develop AML comprising examining a sample from the subject and determining whether there is a positive correlation of expression of miRs with high bone marrow (BM) blasts, the miRs comprising one or more of: hsa-miR-133b, hsa-miR-214, hsa-miR-25, hsa-miR-181a, hsa-miR-181b, hsa-miR-220, hsa-miR-92, hsa-miR-184, hsa-miR-92, hsa-miR-124a, hsa-miR-100, hsa-miR-181b, hsa-miR-135, hsa-miR-155, hsa-miR-222, and hsa-miR-181c.

7. A method for determining diagnosing whether a subject has or will develop AML comprising examining a sample from the subject and determining whether there is a positive correlation of one or more miRs, including one or more of:
miR-155 and miR-181b with the subject's white blood count (for WBC), peripheral and bone marrow blasts percentage;
miR-30b and miR-30c with the subject's white blood count (for WBC) and bone marrow blast percentage, and
miR-25 with the subject's white blood count circulating blast percentages.

8. A miRNA signature associated with a defined cytogenetic subgroup of, 11q23 balanced translocations; comprising one or more miRs from the group of miRs: miR-326, miR-219, miR-194, miR-301, miR-324, miR-339, miR-99b, and miR-328; and one or more down-regulated miRs selected from the group of: miR-34b, miR-15a, miR-29a, miR-29c, miR-372, miR-30a, miR-29b, miR-30e, miR-196a, let-7f, miR-102, miR-331, miR-299, and miR-193.

9. A miRNA signatures associated with a defined cytogenetic subgroup of t(6;11) 11q23 balanced translocations; comprising one or more miRs from the group: hsa-miR-21, hsa-miR-26a, hsa-miR-128b, hsa-miR-130b, hsa-miR-27a, hsa-miR-99, hsa-miR-26b, hsa-miR-23a, hsa-miR-23b, hsa-miR-130a, hsa-miR-24, hsa-miR-30c, hsa-miR-103, hsa-miR-192, hsa-miR-1, and hsa-miR-221.

10. A miRNA signature associated with a defined cytogenetic subgroup of trisomy 8; comprising one or more miRs from the group of: hsa-miR-337, hsa-miR-184, hsa-miR-302b, hsa-miR-105, hsa-let-7d, hsa-miR-1 153, hsa-miR-124a*, hsa-miR-215, hsa-miR-1, hsa-miR-194, hsa-miR-29c, hsa-miR-208, hsa-miR-199a, hsa-miR-24-1, hsa-miR-302c, hsa-miR-367, hsa-miR-200a, hsa-miR-183, hsa-miR-199b, hsa-miR-143, hsa-miR-96, hsa-miR-29b, hsa-miR-202, hsa-miR-340, hsa-miR-102, hsa-miR-191, hsa-let-7i, hsa-miR-30d*, hsa-miR-9-3, hsa-miR-203, hsa-miR-302a, hsa-miR-199a, hsa-miR-206, hsa-miR-197, hsa-miR-198, hsa-miR-372, hsa-miR-182, hsa-miR-193, hsa-miR-325, hsa-miR-192, hsa-miR-204, and hsa-miR-299.

11. A signature in NK-AML composed of miRs comprising up-regulated miRNAs (miR-10a, miR-10b, miR-26a, miR-30c, let-7a-2, miR-16-2, miR-21, miR-181b, miR-368, and miR-192), and down-regulated miRNAs (miR-126, miR-203, miR-200c, miR-182, miR-204, miR-196b, miR-193, miR-191, miR-199a, miR-194, miR-183, miR-299, and miR-145).

12. A miRNA signature associated with a defined cytogenetic subgroup of FLT3-ITD mutations in AML patients, comprising one or more miRs from the group of: miR-155 overexpressed in FLT3-ITD mutations in AML patients: miR-155, miR-10a, and miR-10b.

13. A miRNA signature associated with the outcome, overall survival (OS) an/or event free survival (EFS) in newly diagnosed AML patients, comprising one or more miRs from the group of: miR-199a, miR-199b, miR-191, miR-25, and miR-20a, wherein such overexpression is an indication of an adverse OS.

14. The signature of claim 13, wherein miR-199a and miR-191 are correlated to OS, and/or EFS.

15. (canceled)

16. A miRNA signature of miRs differentially expressed in treated patients with t(11q23) compared with other treated AML patients with other cytogenetic abnormalities including normal karyotype, comprising one or more of the miRs that are up-regulated: hsa-miR-326, hsa-miR-330, hsa-miR-99b, hsa-miR-194, hsa-miR-133b, hsa-miR-339, hsa-miR-138, hsa-miR-128a, hsa-miR-219, hsa-miR-129-2, hsa-miR-138, hsa-miR-210, hsa-miR-301, hsa-miR-200b, hsa-miR-328, and hsa-miR-324.

17. A miRNA signature of miRs differentially expressed in treated patients with t(11q23) compared with other treated AML patients with other cytogenetic abnormalities including normal karyotype, wherein comprising one or more of the miRs that are down-regulated: hsa-miR-29c, hsa-miR-30a-3p, hsa-miR-15a, hsa-miR-29a, hsa-miR-133a, hsa-let-7d, hsa-miR-21, hsa-miR-29b, hsa-miR-370, hsa-miR-34b, hsa-miR-102, hsa-miR-142-5p, hsa-miR-195, hsa-let-7f, hsa-miR-203-prec, hsa-miR-181c, hsa-miR-19b, hsa-miR-194-1, hsa-miR-331-prec, hsa-miR-182*, hsa-miR-183-prec, hsa-miR-16, hsa-miR-302c*, hsa-miR-299-3p, and hsa-miR-30e.

18. (canceled)

19. A miRNA signature of miRs differentially expressed in normal karyotype treated AML patients compared with abnormal karyotype treated AML patients, comprising one or more of the miRs that are up-regulated: hsa-miR-21, hsa-let-7d, hsa-miR-30c, hsa-miR-15b, hsa-miR-219, hsa-miR-302b*, hsa-miR-15a, hsa-miR-34b, hsa-miR-16-1, hsa-miR-16-2, hsa-miR-30e-5p, hsa-miR-140, hsa-miR-15a, hsa-let-7a-2, hsa-miR-30b, hsa-miR-222, hsa-miR-10b, hsa-miR-26a, hsa-miR-10a, hsa-miR-195, hsa-let-7a, and hsa-miR-181b.

20. A miRNA signature of miRs differentially expressed in normal karyotype treated AML patients compared with abnormal karyotype treated AML patients, comprising one or more of the miRs that are down-regulated: hsa-miR-193a, hsa-miR-204, hsa-miR-196a, hsa-miR-205, hsa-miR-200b, hsa-miR-198, hsa-miR-212, hsa-miR-188, hsa-miR-200c, hsa-miR-194, hsa-miR-206, hsa-miR-203, hsa-miR-204-prec, hsa-miR-126, hsa-miR-182*, hsa-miR-199a, hsa-miR-183, hsa-miR-30b, hsa-miR-145, hsa-miR-187, hsa-miR-299-3p, hsa-miR-128a, and hsa-miR-143.

21. A miRNA signature of miRs differentially expressed in treated AML patients with FLT3-ITD mutations vs. FLT3-wt, comprising one or more of the miRs: has-miR-19a, has-miR-155, has-miR-10a, has-miR-99b, and has-miR-192b.

22.-37. (canceled)

38. A method of treating leukemia in a subject, comprising:
determining the amount of at least one biomarker in leukemia cells, relative to control cells; wherein the biomarker is selected from one or more of the miRs, or functional variants therof, listed in: FIG. 5—Table 2, FIG. 8—Table S2, FIG. 9—Table S3, FIG. 10—Table S4, FIG. 11—Table S5, FIG. 12—Table S6, FIG. 14—Table S8, FIG. 15—Table S9 and FIG. 16—Table S10, and
altering the amount of biomarker expressed in the leukemia cells by:
administering to the subject an effective amount of at least one isolated biomarker, if the amount of the biomarker expressed in the cancer cells is less than the amount of the biomarker expressed in control cells; or
administering to the subject an effective amount of at least one compound for inhibiting expression of the at least one biomarker, if the amount of the biomarker expressed in the cancer cells is greater than the amount of the biomarker expressed in control cells.

39. A pharmaceutical composition for treating leukemia, comprising at least one isolated biomarker, wherein the biomarker is selected from one or more of the miRs, or functional variants thereof, listed in: FIG. 5—Table 2, FIG. 8—Table S2, FIG. 9—Table S3, FIG. 10—Table S4, FIG. 11—Table S5, FIG. 12—Table S6, FIG. 14—Table S8, FIG. 15—Table S9 and FIG. 16—Table S10, and a pharmaceutically-acceptable carrier.

40.-41. (canceled)

42. A method of identifying an anti-leukemia agent, comprising providing a test agent to a cell and measuring the level of at least one biomarker associated with increased expression levels in leukemia cells, wherein a decrease in the level of the biomarker in the cell, relative to a control cell, is indicative of the test agent being an anti-cancer agent,
wherein the biomarker is selected from one or more of the miRs, or functional variants thereof, listed in: FIG. 5—Table 2, FIG. 8—Table S2, FIG. 9—Table S3, FIG. 10—Table S4, FIG. 11—Table S5, FIG. 12—Table S6, FIG. 14—Table S8, FIG. 15—Table S9 and FIG. 16—Table S10.

43. A method of assessing the effectiveness of a therapy to prevent, diagnose and/or treat a leukemia associated disease, comprising:
subjecting an animal to a therapy whose effectiveness is being assessed, and
determining the level of effectiveness of the treatment being tested in treating or preventing the disease, by evaluating at least one biomarker,
wherein the biomarker is selected from one or more of the miRs, or functional variants thereof, listed in: FIG. 5—Table 2, FIG. 8—Table S2, FIG. 9—Table S3, FIG. 10—Table S4, FIG. 11—Table S5, FIG. 12—Table S6, FIG. 14—Table S8, FIG. 15—Table S9 and FIG. 16—Table S10.

44-46. (canceled)

47. A kit for screening for a candidate compound for a therapeutic agent to treat a leukemia associated disease, wherein the kit comprises: one or more reagents of at least one biomarker and a cell expressing at least one biomarker,
wherein the biomarker is selected from one or more of the miRs, or functional variants thereof, listed in: FIG. 5—Table 2, FIG. 8—Table S2, FIG. 9—Table S3, FIG. 10—Table S4, FIG. 11—Table S5, FIG. 12—Table S6, FIG. 14—Table S8, FIG. 15—Table S9 and FIG. 16—Table S10.

48.-49. (canceled)

50. A method of treating, preventing, reversing or limiting the severity of a leukemia associated disease complication in an individual in need thereof, comprising: administering to the individual an agent that interferes with at least a leukemia associated disease response cascade, wherein the agent comprises at least one biomarker,
wherein the biomarker is selected from one or more of the miRs, or functional variants thereof, listed in: FIG. 5—Table 2, FIG. 8—Table S2, FIG. 9—Table S3, FIG. 10—Table S4, FIG. 11—Table S5, FIG. 12—Table S6, FIG. 14—Table S8, FIG. 15—Table S9 and FIG. 16—Table S10.

51.-70. (canceled)

71. A composition of matter comprising at least one isolated nucleic acid comprising sense or antisense miR-199a and miR-191.

72. A composition of matter comprising at least one isolated nucleic acid comprising three or more sense or antisense miRs selected from the group consisting of: miR-93; miR-125a; miR-126; miR-130a; and miR-146.

73. A composition of claim 71, comprising at least one isolated nucleic acid comprising five or more sense or antisense miRs selected from the group consisting of: miR-10a; miR-10b; miR-21; miR-25; miR-26; miR-29; miR-30b; miR-30c; miR-93; miR-125a; miR-126; miR-130a; miR-146; miR-155; miR-181b; miR-191; miR-196; miR-199a; and miR-199b.

74. A kit comprising reagents for detecting three or more sense or antisense miRs selected from the group consisting of: miR-93; miR-125a; miR-126; miR-130a; and miR-146.

75. A kit of claim 74, comprising reagents for detecting three or more sense or antisense miRs selected from the group consisting of: miR-10a; miR-10b; miR-21; miR-25; miR-26; miR-29; miR-30b; miR-30c; miR-93; miR-125a; miR-126; miR-130a; miR-146; miR-155; miR-181b; miR-191; miR-196; miR-199a; and miR-199b.

76. A method to affect AML cancer cells comprising:
a. introducing a composition to AML cancer cells, and
b. affecting AML cancer cells,
wherein the composition comprises at least one isolated nucleic acid comprising three or more nucleic acids which comprise sense or antisense miRs selected from the group consisting of: miR-93; miR-125a; miR-126; miR-130a; and miR-146.

77. A method of claim 76, comprising:
a. introducing a composition to AML cancer cells, and
b. affecting AML cancer cells,
wherein the composition comprises at least one isolated nucleic acid comprising five or more nucleic acids which comprise sense or antisense miRs selected from the group consisting of: miR-10a; miR-10b; miR-21; miR-25; miR-26; miR-29; miR-30b; miR-30c; miR-93; miR-125a; miR-126; miR-130a; miR-146; miR-155; miR-181b; miR-191; miR-196; miR-199a; and miR-199b.

78. A method of claim 76, comprising:
a. introducing a test compound and a composition to AML cancer cells, and
b. identifying test compounds useful to affect AML cancer cells,
wherein the composition comprises at least one isolated nucleic acid comprising three or more sense or antisense miRs selected from the group consisting of: miR-93; miR-125a; miR-126; miR-130a; and miR-146.

79. A method of claim 76, comprising:
a. introducing a test compound and a composition to AML cancer cells, and
b. identifying test compounds useful to affect AML cancer cells
wherein the composition comprises at least one isolated nucleic acid comprising five or more sense or antisense miRs selected from the group consisting of: miR-10a; miR-10b; miR-21; miR-25; miR-26; miR-29; miR-30b; miR-30c; miR-93; miR-125a; miR-126; miR-130a; miR-146; miR-155; miR-181b; miR-191; miR-196; miR-199a; and miR-199b.

80. A method to identify useful AML cancer therapeutic compounds, comprising
a. correlating a miR fingerprint of cells exposed to a test compound with control, and
b. identifying useful AML cancer therapeutic compounds,
wherein the control comprises a miR fingerprint comprising three or more markers selected from the group consisting of: underexpressed miR-93; underexpressed miR-125a; underexpressed miR-126; underexpressed miR-130a; and underexpressed miR-146.

81. A method of claim 80, comprising:
a. correlating a miR fingerprint of cells exposed to a test compound with control, and
b. identifying useful AML cancer therapeutic compounds,
wherein the control comprises a miR fingerprint comprising five or more markers selected from the group consisting of: overexpressed miR-10a; overexpressed miR-10b; overexpressed miR-21; overexpressed miR-25; overexpressed miR-26; underexpressed miR-29; overexpressed miR-30b; overexpressed miR-30c; underexpressed miR-93; underexpressed miR-125a; underexpressed miR-126; underexpressed miR-130a; underexpressed miR-146; overexpressed miR-155; overexpressed miR-181b; overexpressed miR-191; underexpressed miR-196; overexpressed miR-199a; and overexpressed miR-199b.

82. A method to identify or predict AML cell status, comprising:
a. correlating a miR fingerprint in a cell-containing test sample with control, and
b. identifying or predicting AML cell status,
wherein the control comprises a miR fingerprint comprising three or more markers selected from the group consisting of: underexpressed miR-93; underexpressed miR-125a; underexpressed miR-126; underexpressed miR-130a; and underexpressed miR-146.

83. A method of claim 82, comprising:
a. correlating a miR fingerprint in a cell-containing test sample with control, and
b. identifying or predicting AML cell status,
wherein the control comprises a miR fingerprint comprising five or more markers selected from the group consisting of: overexpressed miR-10a; overexpressed miR-10b; overexpressed miR-21; overexpressed miR-25; overexpressed miR-26; underexpressed miR-29; overexpressed miR-30b; overexpressed miR-30c; underexpressed miR-93; underexpressed miR-125a; underexpressed miR-126; underexpressed miR-130a; underexpressed miR-146; overexpressed miR-155; overexpressed miR-181b; overexpressed miR-191; underexpressed miR-196; overexpressed miR-199a; and overexpressed miR-199b.

84. A method to identify or predict human AML cancer status, comprising:
a. correlating a miR fingerprint in a human-derived test sample with control, and
b. identifying or predicting human AML cancer status,
wherein the control comprises a miR fingerprint comprising three or more markers selected from the group consisting of: underexpressed miR-93; underexpressed miR-125a; underexpressed miR-126; underexpressed miR-130a; and underexpressed miR-146.

85. A method of claim 84, comprising:
a. correlating a miR fingerprint in a cell-containing test sample with control, and
b. identifying or predicting human AML cancer status,
wherein the control comprises a miR fingerprint comprising five or more markers selected from the group consisting of: overexpressed miR-10a; overexpressed miR-10b; overexpressed miR-21; overexpressed miR-25; overexpressed miR-26; underexpressed miR-29; overexpressed miR-30b; overexpressed miR-30c; underexpressed miR-93; underexpressed miR-125a; underexpressed miR-126; underexpressed miR-130a; underexpressed miR-146; overexpressed miR-155; overexpressed miR-181b; overexpressed miR-191; underexpressed miR-196; overexpressed miR-199a; and overexpressed miR-199b.

86. A method to ameliorate AML cancer in a human in need of such amelioration, comprising:
a. administering a AML cancer-ameliorating therapeutic to a human having AML cancer, and
b. ameliorating the AML cancer,
wherein the therapeutic comprises at least one isolated nucleic acid comprising three or more antisense miRs selected from the group consisting of: miR-93; miR-125a; miR-126; miR-130a; and miR-146.

87. A method of claim 86, comprising:
a. administering a AML cancer-ameliorating therapeutic to a human having AML cancer, and
b. ameliorating the AML cancer,
wherein the therapeutic comprises at least one isolated nucleic acid comprising five or more miRs selected from the group consisting of: antisense miR-10a; antisense miR-10b; antisense miR-21; miR-25; antisense miR-26; miR-29; miR-30b; miR-30c; miR-93; miR-125a; miR-126; miR-130a; miR-146; antisense miR-155; antisense miR-181b; antisense miR-191; miR-196; antisense miR-199a; and antisense miR-199b.

88. A method to predict human AML cancer survival, comprising:
a. correlating a miR fingerprint in a human AML cell-containing test sample with control, and
b. predicting human AML cancer survival,
wherein the control comprises a miR fingerprint comprising two or more expression markers selected from the group consisting of: miR-191; miR-199a; and miR-199b.

89. A method of claim 88, comprising:
a. correlating a miR fingerprint in a human AML cell-containing test sample with control, and
b. identifying or predicting human FLT3-ITD+ AML cancer status,
wherein the control comprises a miR fingerprint comprising two or more expression markers selected from the group consisting of: miR-10a; miR-10b; and miR-155.

90. A method of claim 88, comprising:
a. correlating a miR fingerprint in a human AML cell-containing test sample with control, and
b. identifying human AML cancer white blood cell or marrow cell status,
wherein the control comprises a miR fingerprint comprising two or more expression markers selected from the group consisting of: miR-25; miR-30b; miR-30c; miR-155; and miR-181b.

91. A method of claim 88, comprising:
a. correlating a miR fingerprint in a human AML cell-containing test sample with control, and
b. identifying or predicting human AML cancer cytogenetic status,
wherein the control comprises a miR fingerprint comprising two or more expression markers selected from the group consisting of: miR-21; miR-26; miR-29; and miR-196.

92. A method of claim 88, comprising:
a. correlating a miR fingerprint in a human AML cell-containing test sample with control, and
b. identifying or predicting human AML cancer multilineage dysplasia status,
wherein the control comprises a miR fingerprint comprising miR-181a.
